(19) United States
(12) Patent Application Publication
(10) Pub. No.: US 2011/0027379 A1
(43) Pub. Date: Feb. 3, 2011

(54) Oligo-Ethylene Glycol-Based Polymer Compositions and Methods of Use

(75) Inventors: Chih-Chang Chu, Ithaca, NY (US); Ramaz Katsarava, Tbilisi (GE); Zaza D. Gomurashvili, La Jolla, CA (US); William G. Turnell, Del Mar, CA (US)

Correspondence Address: DLA PIPER LLP (US), 4365 EXECUTIVE DRIVE, SUITE 1100, SAN DIEGO, CA, 92121-2133, US

(73) Assignee: Cornell University

(21) Appl. No.: 12/746,500

(22) PCT Filed: Dec. 4, 2008
(86) PCT No.: PCT/US08/85583

§ 371 Date: Oct. 22, 2010

Related U.S. Application Data
(60) Provisional application No. 60/992,950, filed on Dec. 6, 2007.

(51) Int. Cl. A61K 9/14; A61K 47/34; C08G 63/685; B32B 5/00; A61P 35/00; A61P 29/00; B82Y 5/00

(52) U.S. Cl. 424/501; 514/772.3; 528/290; 428/402; 977/773

(57) ABSTRACT

The invention provides biodegradable PEAs, PEURs and PEUs that are synthesized by solution polycondensation to include α-amino acids and oligo-ethylene ether segments in the polymer backbone. The polymers can be obtained by substituting oligo-ethylene glycol (OEG) for aliphatic di-acid and diols during their fabrication. Also provided are compositions in which bioactive agents are dispersed in the polymers. The compositions biodegrade by enzymatic action to release incorporated bioactive agents and oligo-ethylene glycol segments, which are fully biodegradable at a molecular weight less than 400 Da. Due to their comparatively rapid surface enzymatic hydrolysis, the compositions can be used to deliver bioactive agents in a controlled manner within a relatively rapid delivery time, such as about 18 to 24 hours.

### FIELD OF THE INVENTION

The invention relates, in general, to drug delivery systems and, in particular, to polymer compositions that can deliver a variety of different types of molecules in a controlled time release fashion.

### BACKGROUND INFORMATION

Attempts have been made using different methods to develop compositions that control the delivery profile of drugs, either for sustained delivery or for rapid, but smooth. release, such as for delivery of cancer treatment and pain relief drugs. For example, such compositions as micro/nanospheres, liposomes, albumin conjugates, water-soluble prodrugs, cyclodextrin complexes and hydrogels have been attempted for such purposes, but with limited success due to a tendency of such compositions to release a large initial burst of drug with consequent failure of sustained delivery for a controlled time period.

In one technique that has been studied, bioactive agents, including peptides, have been conjugated to polyethylene glycol (PEG) to increase the half-life of the bioactive agent. The delivery profiles of such conjugates generally show that the half-life of the drug in circulation is proportional to the molecular weight of the PEG molecule used. The biokinetics and biodistribution of PEG have been shown to depend as well upon the size of the PEG molecules used.

Despite these advances in the art, the need exists for new and better compositions and methods for use of poly-ethylene glycol and molecules of similar chemical structure for administration of various bioactive agents, for example, to achieve a smooth release rate profile with sustained delivery for a controlled period of time.

### SUMMARY OF THE INVENTION

The present invention is based on the premise that oligo-ethylene glycol-containing (OEG-based) molecules can be introduced into the polymer backbone of poly(ester amide) (PEA), poly(ester urethane) (PEUR), and poly(ester urea) (PEU) polymers that contain at least one α-amino acid in the polymer backbone per repeat unit. Such oligo-ethylene glycol-containing polymers are referred to herein as poly(ester ether amide) (PEEA), poly(ester ether urethane) (PEEUR) and poly(ester ether urea) (PEEU) and can be used to formulate biodegradable polymer compositions for rapid release of one or more dispersed bioactive agents in a consistent and reliable manner. For example, such compositions can be used to release a bioactive agent contained therein with a smooth release rate profile within a period of about 24 hours.

Accordingly, in one embodiment, the invention provides an OEG-based composition in which a bioactive agent is dispersed in a biodegradable polymer. The polymer contains at least one of the following a) through f):

a) a poly(ester ether amide) (PEEA) having a chemical formula described by structural formula (I),

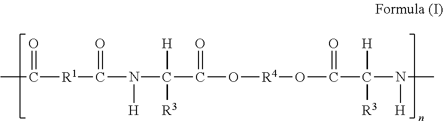

wherein n ranges from about 15 to about 150;

wherein, R¹ is independently selected from the group consisting of (C₂-C₁₂) alkylene, (C₂-C₁₂) alkenylene, and residues of α,ω-dicarboxylates of formula (II), wherein R⁵ in formula (II) is independently selected from the group consisting of (C₂-C₄) alkylene and (C₂-C₄) alkenylene and R⁷ is independently selected from the group consisting of (C₂-C₆) alkyloxy (C₂-C₁₂) alkylene, except that at least one R¹ in each polymer is the residue of a α,ω-dicarboxylate of formula (II) wherein R⁷ is (C₂-C₆) alkyloxy (C₂-C₁₂) alkylene;

the R³s in individual n monomers are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂SCH₃; and

R⁴ is independently selected from (C₂-C₆) alkyloxy (C₂-C₁₂) alkylene, (C₂-C₂₀) alkylene, (C₂-C₂₀) alkenylene, CH₂CH(OH)CH₂, CH₂CH(CH₂OH), a bicyclic-fragment of a 1,4:3,6-dianhydrohexitol of structural formula (III), a fragment of 1,4-anhydroerythritol, and combinations thereof;

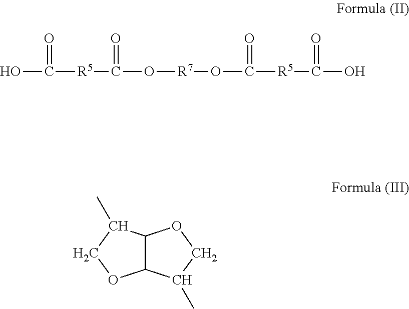

b) a PEEA polymer having a chemical formula described by structural formula (IV):

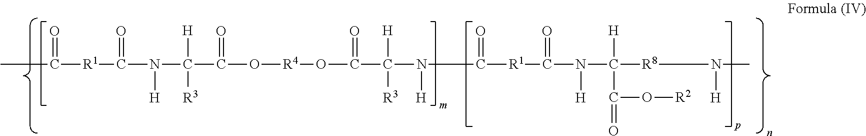

wherein n ranges from about 15 to about 150, m ranges about 0.1 to 0.9: p ranges from about 0.9 to 0.1;

wherein R¹ is independently selected from the group consisting of (C₂-C₁₂) alkylene, (C₂-C₁₂) alkenylene, and residues of α,ω-dicarboxylates of formula (II), wherein R⁵ in formula (II) is independently selected from the group consisting of (C₂-C₄) alkylene and (C₂-C₄) alkenylene and R⁷ is independently selected from the group consisting of (C₂-C₆) alkyloxy (C₂-C₁₂) alkylene, except that at least one R¹ in each polymer is the residue of a α,ω-dicarboxylate of formula (II)) wherein R⁷ is (C₂-C₆) alkyloxy (C₂-C₁₂) alkylene;

R² is independently selected from the group consisting of hydrogen, (C₁-C₁₂) alkyl, (C₆-C₁₀) aryl (C₁-C₆) alkyl or a protecting group;

R³s in individual m monomers are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂SCH₃;

R⁴ is independently selected from (C₂-C₆) alkyloxy (C₂-C₁₂) alkylene, (C₂-C₂₀) alkylene, (C₂-C₂₀) alkenylene, CH₂CH(OH)CH₂, CH₂CH(CH₂OH), a bicyclic-fragment of a 1,4:3,6-dianhydrohexitol of structural formula (III), a fragment of 1,4-anhydroerythritol, and combinations thereof; and

R⁸ is independently (C₁-C₂₀) alkyl or (C₂-C₂₀) alkenyl;

c) a poly(ether urethane) (PEEUR) having a chemical formula described by structural formula (V),

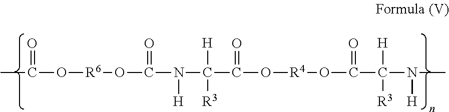

wherein, n ranges from about 15 to about 150;

R³s within an individual n monomer are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂SCH₃; and

R⁴ and R⁶ are independently selected from the group consisting of (C₂-C₆) alkyloxy (C₂-C₁₂) alkylene, CH₂CH(OH)CH₂, CH₂CH(CH₂OH), a bicyclic fragment of a 1,4:3,6-dianhydrohexitols of structural formula (III), a fragment of 1,4-anhydroerythritol and combinations thereof, except that at least one of R⁴ and R⁶ in each polymer is selected from the group consisting of (C₂-C₆) alkyloxy (C₂-C₁₂) alkylene;

d) a PEEUR having a chemical structure described by general structural formula (VI)

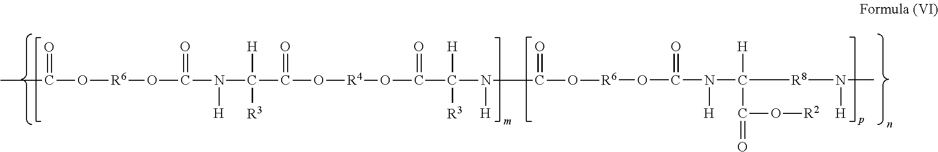

wherein n ranges from about 15 to about 150, m ranges about 0.1 to about 0.9, p ranges from about 0.9 to about 0.1;

R² is independently selected from the group consisting of hydrogen, (C₁-C₁₂) alkyl, (C₁-C₆) alkyl (C₆-C₁₀) aryl, and a protecting group;

R³s within an individual m monomer are independently selected from the group consisting of hydrogen, (C₁-C₆) alkyl, (C₂-C₆) alkenyl, (C₂-C₆) alkynyl, (C₆-C₁₀) aryl(C₁-C₆) alkyl, and —(CH₂)₂SCH₃;

R⁴ and R⁶ are independently selected from the group consisting of (C₂-C₆) alkyloxy (C₂-C₁₂) alkylene, CH₂CH(OH)CH₂, CH₂CH(CH₂OH), a bicyclic fragment of a 1,4:3,6-dianhydrohexitol of structural formula (III), a fragment of 1,4-anhydroerythritol and combinations thereof, except that at least one of R⁴ and R⁶ in each polymer is selected from the group consisting of (C₂-C₆) alkyloxy (C₂-C₁₂) alkylene; and

R⁸ is independently (C₁-C₂₀) alkyl or (C₂-C₂₀) alkenyl;

e) a poly(ether urea) (PEEU) having a chemical formula described by general structural formula (VII):

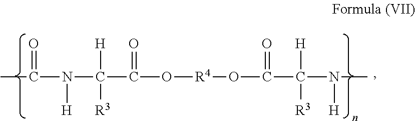

wherein n is about 15 to about 150;

R³s within an individual n monomer are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂SCH₃; and

R⁴ is independently selected from the group consisting of (C₂-C₆) alkyloxy (C₂-C₁₂) alkylene, CH₂CH(OH)CH₂, CH₂CH(CH₂OH), a bicyclic fragment of a 1,4:3,6-dianhydrohexitol of structural formula (III), a fragment of 1,4-anhydroerythritol, and combinations thereof, except that at least one R⁴ in each polymer is selected from the group consisting of (C₂-C₆) alkyloxy (C₂-C₁₂) alkylene; and

f) Formula VIII is a PEEU having a chemical formula described by structural formula (VIII)

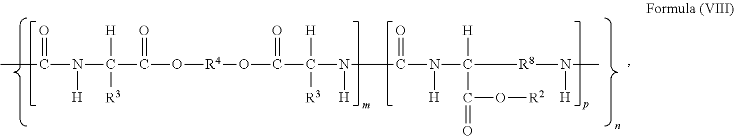

wherein m is about 0.1 to about 1.0; p is about 0.9 to about 0.1; n is about 15 to about 150;

R² is independently the group consisting of hydrogen, (C₁-C₁₂) alkyl or (C₆-C₁₀) aryl;

R³s within an individual m monomer are independently selected from the group consisting of hydrogen, (C₁-C₆) alkyl, (C₂-C₆) alkenyl, (C₂-C₆) alkynyl, (C₆-C₁₀) aryl (C₁-C₆)alkyl, and —(CH₂)₂SCH₃;

R⁴ is independently selected from the group consisting of (C₂-C₆) alkyloxy (C₂-C₁₂) alkylene, CH₂CH(OH)CH₂, CH₂CH(CH₂OH), a bicyclic fragment of a 1,4:3,6-dianhydrohexitol of structural formula (III), a fragment of 1,4-anhydroerythritol and combinations thereof, except that at least one R⁴ in each polymer is selected from the group consisting of (C₂-C₆) alkyloxy (C₂-C₁₂) alkylene; and

R⁸ is independently (C₁-C₂₀) alkyl or (C₂-C₂₀) alkenyl.

In another embodiment, the invention provides a biodegradable OEG-based polymer having a chemical structure described by Formula I, IV, V, VI, VII, or VIII, above.

In yet another embodiment, the invention provides biodegradable micelle-forming polymer compositions useful for delivery of bioactive agents dispersed therein. The composition includes a polymer with repeating alternating units of a) a hydrophobic section containing at least one invention biodegradable OEG-based polymer having a chemical structure described by structural formula I, IV, V, VI, VII, or VIII, above joined to b) a water soluble section. The water soluble section is made of repeating alternating units of i) polyethylene glycol having a Mw of 400 Dalton to about 200 Dalton, and ii) at least one ionizable or polar amino acid. The repeating alternating units have substantially similar molecular weights and the molecular weight of the polymer is in the range from about 15 kDa to 300 kDa.

In still another embodiment, the invention provides methods for delivering a bioactive agent to a subject by administering to the subject in vivo an invention OEG-based polymer composition containing at least one bioactive agent dispersed in at least one or a blend of the invention OEG-based biodegradable polymers having chemical structures described by structural formula structural formula I, IV, V, VI, VII, or VIII, above. The composition can be formulated as a liquid dispersion of polymer particles with at least one bioactive agent disbursed therein. The particles biodegrade by enzymatic action at the surface thereof to release the bioactive agent with substantially zero-order release kinetics over a period of about 24 hours.

### A BRIEF DESCRIPTION OF THE FIGURES

FIG. 1 is a graph showing the effect of enzyme (a-chymotrypsin) concentration on the weight loss kinetics of invention OEG-based PEA, AP3EG, at 37° C., and pH 7.4. PBS buffer serves as the control. (-▪-)=PBS buffer; (--)=α-chymotrypsin at 0.05 mg/mL; (-▴-)=α-chymotrypsin at 0.10 mg/mL; (-569 -)=α-chymotrypsin at 0.20 mg/mL.

FIG. 2 is a graph showing the effect of diacid structure (Monomer 1) incorporated into an invention PEEA upon its weight loss in PBS or α-chymotrypsin medium (0.1 mg/mL) at 37° C. Solid line=enzyme solution. Dashed line=PBS medium.

-▪-=Monomer 1a (di-p-nitrophenyl adipate with 4 methylene groups); -∘-=Monomer 1b (di-p-nitrophenyl sebacate with 8 methylene groups).

FIGS. 3A-C are graphs showing in vitro lipase catalyzed biodegradation as compared with that in pure phosphate buffered medium in terms of weight loss in mg/cm² of three invention PEEURs as compared with that of PEA 8-L-Leu-6, which contains no OEG segment. FIG. 3A=EG2-Leu-2; FIG. 3B=EG3-Leu-2; FIG. 3C=EG4-Leu-2. Curve a) lipase catalyzed phosphate buffered medium with pH 7.4, t=37° C.; curve b) pure phosphate buffered medium; curve c) degradation of PEA 8-L-Leu-6 in lipase catalyzed phosphate buffered medium.

### DETAILED DESCRIPTION OF THE INVENTION

The invention is based on the discovery that α-amino acid-containing PEA, PEUR and PEU polymers that contain backbone segments having one or more ester linkages between the amino acids are fully biodegradable so long as the OEG-based backbone segments upon biodegradation form OEGs with a molecular weight (Mw) in the range from about 44 (monoethyleneglycol) up to, but not including 400 Da. The at least one ester linkage in the polymer backbone is readily introduced in the diol components (and the di-acids in the case of PEEAs) used in polycondensation of the invention family of polymers. Due to structural properties of the invention PEEA, PEEUR and PEEU polymers, compositions made using such polymers can be tailored to achieve a release rate of disbursed bioactive agents suitable to meet various therapeutic goals. For example, particles and films made using such polymers can be designed to biodegrade with a release rate similar to a twenty four hour infusion without a large initial burst of drug release and having a substantially zero-order release profile.

More particularly, in one embodiment, the invention provides biodegradable polymer compositions containing at least one bioactive agent disbursed in at least one of a family of biodegradable polymers, which are referred to herein as poly(ether ester amide)s (PEEAs), poly(ether ester urethane)s (PEEURs) and poly(ether ester urea)s (PEEUs). The polymers used in the invention compositions contain at least one OEG-based backbone segment, such that, upon biodegradation, the OEG-based backbone segments are released with a molecular weight (Mw) of 400 Da or less. Therefore, upon biodegradation, the polymers, including the OEG-based segments thereof, are fully biodegradable without formation of irritants (R. Mehvar, J. Pharm Pharmaceut Sci (2000) 3 (1): 125-136; and T. Yamaoka, J. Pharm Sci (1994) Apr, 83(4):601-6). In addition, because OEG segments of 400 Da or less are screened from the circulation by the endoreticular and renal system in mammals, the polymer degradation products and any bioactive agents that may have been dispersed in the polymers are cleared from the circulation upon enzymatic biodegradation.

In one embodiment, therefore, the invention PEEA, PEEUR and PEEU polymers are made by using at least one diol (or di-acid in the case of PEEAs) containing OEG-based segments with molecular weights of 400 Da or less during fabrication of the polymers. If only such diols and di-acids are used in fabrication, the PEEA, PEEUR and PEEU polymers produced are analogs of known PEG-containing polymers, but upon biodegradation the major breakdown products are α-amino acids, such as biological α-amino acids, and OEG segments of 44 up to, but less than 400 Da. Hence, the invention polymers and compositions based thereon are readily biodegraded by mammalian subjects without the irritation that results from break down of polymers containing OEG segments of greater molecular weight, such as is the case with polymers that form PEG breakdown products (i.e., those with a Mw of 400 Da or greater).

More particularly, in one embodiment, the invention provides a composition comprising at least one bioactive agent dispersed in a biodegradable OEG-based polymer comprising at least one or a blend of the following a) through f):

a) a poly(ester ether amide) (PEEA) having a chemical formula described by structural formula (I),

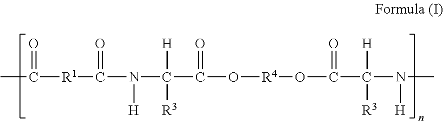

wherein n ranges from about 15 to about 150;

wherein, R¹ is independently selected from the group consisting of (C₂-C₁₂) alkylene, (C₂-C₁₂) alkenylene, and residues of α,ω-dicarboxylates of formula (II), wherein R⁵ in formula (II) is independently selected from the group consisting of (C₂-C₄) alkylene and (C₂-C₄) alkenylene and R⁷ is independently selected from the group consisting of (C₂-C₆) alkyloxy (C₂-C₁₂) alkylene, except that at least one R¹ in each polymer is the residue of a α,ω-dicarboxylate of formula (II) wherein R⁷ is (C₂-C₆) alkyloxy (C₂-C₁₂) alkylene;

the R³s in individual n monomers are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂SCH₃; and

R⁴ is independently selected from (C₂-C₆) alkyloxy (C₂-C₁₂) alkylene, (C₂-C₂₀) alkylene, (C₂-C₂₀) alkenylene, CH₂CH(OH)CH₂, CH₂CH(CH₂OH), a bicyclic-fragment of a 1,4:3,6-dianhydrohexitol of structural formula (III), a fragment of 1,4-anhydroerythritol, and combinations thereof;

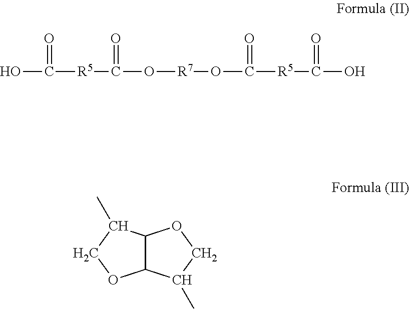

b) a PEEA polymer having a chemical formula described by structural formula (IV):

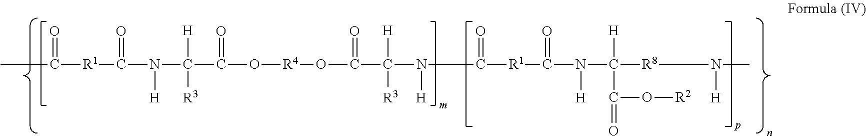

wherein n ranges from about 15 to about 150, m ranges about 0.1 to 0.9: p ranges from about 0.9 to 0.1;

wherein R¹ is independently selected from the group consisting of (C₂-C₁₂) alkylene, (C₂-C₁₂) alkenylene, and residues of α,ω-dicarboxylates of formula (II), wherein R⁵ in formula (II) is independently selected from the group consisting of (C₂-C₄) alkylene and (C₂-C₄) alkenylene and R⁷ is independently selected from the group consisting of (C₂-C₆) alkyloxy (C₂-C₁₂) alkylene, except that at least one R¹ in each polymer is the residue of a α,ω-dicarboxylate of formula (II)) wherein R⁷ is (C₂-C₆) alkyloxy (C₂-C₁₂) alkylene;

R² is independently selected from the group consisting of hydrogen, (C₁-C₁₂) alkyl, (C₆-C₁₀) aryl (C₁-C₆) alkyl or a protecting group;

R³s in individual m monomers are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂SCH₃;

R⁴ is independently selected from (C₂-C₆) alkyloxy (C₂-C₁₂) alkylene, (C₂-C₂₀) alkylene, (C₂-C₂₀) alkenylene, CH₂CH(OH)CH₂, CH₂CH(CH₂OH), a bicyclic-fragment of a 1,4:3,6-dianhydrohexitol of structural formula (III), a fragment of 1,4-anhydroerythritol, and combinations thereof; and

R⁸ is independently (C₁-C₂₀) alkyl or (C₂-C₂₀) alkenyl;

c) a poly(ether urethane) (PEEUR) having a chemical formula described by structural formula (V),

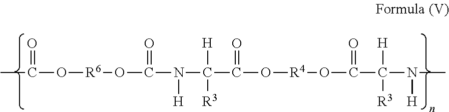

wherein, n ranges from about 15 to about 150;

R³s within an individual n monomer are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂SCH₃; and

R⁴ and R⁶ are independently selected from the group consisting of (C₂-C₆) alkyloxy (C₂-C₁₂) alkylene, CH₂CH(OH)CH₂, CH₂CH(CH₂OH), a bicyclic fragment of a 1,4:3,6-dianhydrohexitols of structural formula (III), a fragment of 1,4-anhydroerythritol and combinations thereof, except that at least one of R⁴ and R⁶ in each polymer is selected from the group consisting of (C₂-C₆) alkyloxy (C₂-C₁₂) alkylene;

d) a PEEUR having a chemical structure described by general structural formula (VI)

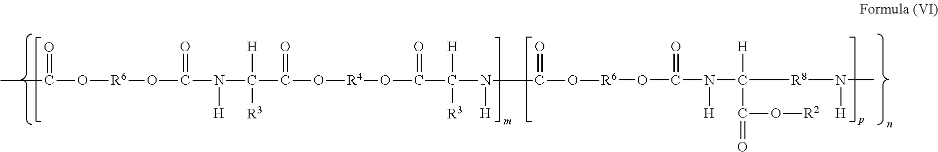

wherein n ranges from about 15 to about 150, m ranges about 0.1 to about 0.9, p ranges from about 0.9 to about 0.1;

R² is independently selected from the group consisting of hydrogen, (C₁-C₁₂) alkyl, (C₁-C₆) alkyl (C₆-C₁₀) aryl, and a protecting group;

R³s within an individual m monomer are independently selected from the group consisting of hydrogen, (C₁-C₆) alkyl, (C₂-C₆) alkenyl, (C₂-C₆) alkynyl, (C₆-C₁₀) aryl(C₁-C₆) alkyl, and —(CH₂)₂SCH₃;

R⁴ and R⁶ are independently selected from the group consisting of (C₂-C₆) alkyloxy (C₂-C₁₂) alkylene, CH₂CH(OH)CH₂, CH₂CH(CH₂OH), a bicyclic fragment of a 1,4:3,6-dianhydrohexitol of structural formula (III), a fragment of 1,4-anhydroerythritol and combinations thereof, except that at least one of R⁴ and R⁶ in each polymer is selected from the group consisting of (C₂-C₆) alkyloxy (C₂-C₁₂) alkylene; and

R⁸ is independently (C₁-C₂₀) alkyl or (C₂-C₂₀) alkenyl;

e) a poly(ether urea) (PEEU) having a chemical formula described by general structural formula (VII):

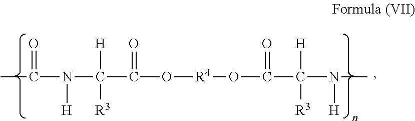

wherein n is about 15 to about 150;

R³s within an individual n monomer are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂SCH₃; and

R⁴ is independently selected from the group consisting of (C₂-C₆) alkyloxy (C₂-C₁₂) alkylene, CH₂CH(OH)CH₂, CH₂CH(CH₂OH), a bicyclic fragment of a 1,4:3,6-dianhydrohexitol of structural formula (III), a fragment of 1,4-anhydroerythritol, and combinations thereof, except that at least one R⁴ in each polymer is selected from the group consisting of (C₂-C₆) alkyloxy (C₂-C₁₂) alkylene; and

f) a PEEU having a chemical formula described by structural formula (VIII)

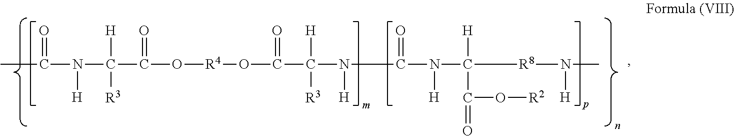

wherein m is about 0.1 to about 1.0; p is about 0.9 to about 0.1; n is about 15 to about 150;

R² is independently the group consisting of hydrogen, (C₁-C₁₂) alkyl or (C₆-C₁₀) aryl;

R³s within an individual m monomer are independently selected from the group consisting of hydrogen, (C₁-C₆) alkyl, (C₂-C₆) alkenyl, (C₂-C₆) alkynyl, (C₆-C₁₀) aryl (C₁-C₆)alkyl, and —(CH₂)₂SCH₃;

R⁴ is independently selected from the group consisting of (C₂-C₆) alkyloxy (C₂-C₁₂) alkylene, CH₂CH(OH)CH₂, CH₂CH(CH₂OH), a bicyclic fragment of a 1,4:3,6-dianhydrohexitol of structural formula (III), a fragment of 1,4-anhydroerythritol and combinations thereof, except that at least one R⁴ in each polymer is selected from the group consisting of (C₂-C₆) alkyloxy (C₂-C₁₂) alkylene; and

R⁸ is independently (C₁-C₂₀) alkyl or (C₂-C₂₀) alkenyl.

For example, in one embodiment in the polymers described by structural formulas (I) or (IV), or in both, the R⁷ in each n monomer is (C₂-C₆) alkyloxy (C₂-C₁₂) alkylene and R⁴ is selected from the group consisting of CH₂CH(OH)CH₂, CH₂CH(CH₂OH) and any one of (C₂-C₆) alkyloxy (C₂-C₁₂) alkylene. In this embodiment, the polymer will biodegrade to form from 15 to 300 OEGs having Mw of 44 up to, but not including, 400 Da.

In yet another embodiment, in the polymer described by structural formula (V) or (VI), or both, R⁴ or R⁶ in each n monomer is selected from the group consisting of CH₂CH(OH)CH₂, CH₂CH(CH₂OH) and (C₂-C₆) alkyloxy (C₂-C₁₂) alkylene. Alternatively, in the polymers in the composition described by structural formula (V) or (VI), or both, both R⁴ and R⁶ in each n monomer are selected from the group consisting of CH₂CH(OH)CH₂, CH₂CH(CH₂OH) and (C₂-C₄) alkyloxy (C₂-C₈) alkylene. In the latter embodiment, the polymer will biodegrade to form from 15 to 300 OEGs having Mw of 44 up to, but not including, 400 Da.

In still another embodiment wherein the polymer in the composition is described by structural formula (VII) or (VIII), or both, R⁴ in each n monomer is selected from the group consisting of CH₂CH(OH)CH₂, CH₂CH(CH₂OH), and (C₂-C₄) alkyloxy (C₂-C₈) alkylene. In this embodiment, the polymer will biodegrade to form from 15 to 300 OEGs having Mw of 44 up to, but not including, 400 Da.

As used herein, “alkenyl” refers to straight or branched chain hydrocarbyl groups having one or more carbon-carbon double bonds.

As used herein, “alkynyl” refers to straight or branched chain hydrocarbyl groups having at least one carbon-carbon triple bond.

As used herein, “aryl” refers to aromatic groups having in the range of 6 up to 14 carbon atoms.

The PEEA, PEEUR and PEEU polymers used in the invention compositions are poly-condensates. The ratios “m” and “p” in Formulas (IV, VI and VIII) are defined as irrational numbers in the description of these poly-condensate polymers. Moreover, as “m” and “p” will each take up a range within any polycondensate, such a range cannot be defined by a pair of integers. Each polymer chain is a string of monomer residues linked together by the rule that all bis-amino acyl diol-diester (i) and adirectional amino acid (e.g. lysine) monomer residues (ii) are linked either to themselves or to each other by a diacid monomer residue (iii) for PEEA, by a diol residue (iii) for PEEUR or carbonyl (iii) for PEEU. Thus, only linear combinations of i-iii-i, i-iii-ii (or ii-iii-i) and ii-iii-ii are formed. In turn, each of these combinations is linked either to themselves or to each other by a diacid monomer residue (iii) for PEEA or a diol residue (iii) for PEEUR or carbonyl (iii) for PEEU. Each polymer chain is therefore a statistical, but non-random, string of monomer residues composed of integer numbers of monomers, i, ii and iii. However, in general for polymer chains of any practical average molecular weight (i.e., sufficient mean length), the ratios of monomer residues “m” and “p” in formulas (IV, VI and VIII) will not be whole numbers (rational integers). Furthermore, for the condensate of all poly-dispersed copolymer chains, the numbers of monomers i, ii and iii averaged over all of the chains (i.e. normalized to the average chain length) will not be integers. It follows that the ratios can only take irrational values (i.e., any real number that is not a rational number). Irrational numbers, as the term is used herein, are derived from ratios that are not of the form n/j, where n and j are integers.

As used herein, the terms “amino acid” and “α-amino acid” mean a chemical compound containing an amino group, a carboxyl group and a pendent R group, such as the R³ groups defined herein. As used herein, the term “biological α-amino acid” means the amino acid(s) used in synthesis are selected from phenylalanine, leucine, glycine, alanine, valine, isoleucine, methionine, or a mixture thereof. As used herein, the term “adirectional amino acid” means a chemical moiety within the polymer chain obtained from an α-amino acid, such that the R group (for example R⁸ in formulas VI and VIII) is inserted within the polymer backbone.

As used herein the term “bioactive agent” means a bioactive agent as disclosed herein that is dispersed in the polymer of the invention composition. As used herein, the term “dispersed” is used to refer to bioactive agents means that the bioactive agent is mixed into, dissolved in, homogenized with, and/or covalently bound to a polymer, for example, attached to a functional group in the polymer of the composition or to the surface of a polymer particle. Such bioactive agents may include, without limitation, small molecule drugs, peptides, proteins, DNA, cDNA, RNA, sugars, lipids and whole cells. The bioactive agents can be administered in polymer particles having a variety of sizes and structures suitable to meet differing therapeutic goals and routes of administration.

The term, “biodegradable” as used herein to describe the invention OEG-based polymer compositions means the polymer used therein is capable of being broken down into innocuous products in the normal functioning of the body. This is particularly true when the amino acids used in fabrication of the invention polymers are biological L-α-amino acids and the diol and di-acids used in fabrication of the polymer form OEG segments of 44 up to, but not including 400 Da upon biodegradation. The polymers in the invention OEG-based polymer compositions include hydrolyzable ester and enzymatically cleavable amide linkages that provide biodegradability, and are typically chain terminated, predominantly with amino groups. Optionally, the amino termini of the polymers can be acetylated or otherwise capped by conjugation to any other acid-containing, biocompatible molecule, to include without restriction organic acids, bioinactive biologics, and bioactive agents as described herein. In one embodiment, the entire polymer composition, and any particles made thereof, is substantially biodegradable.

In one alternative, at least one of the α-amino acids used in fabrication of the invention OEG-based polymers is a biological α-amino acid. For example, when the R³s are CH₂Ph, the biological α-amino acid used in synthesis is L-phenylalanine. In alternatives wherein the R³s are CH₂CH(CH₃)₂, the polymer contains the biological α-amino acid, L-leucine. By varying the R³s within monomers as described herein, other biological α-amino acids can also be used, e.g., glycine (when the R³s are H), alanine (when the R³s are CH₃), valine (when the R³s are CH(CH₃)₂, isoleucine (when the R³s are CH(CH₃)CH₂CH₃), phenylalanine (when the R³s are CH₂C₆H₅), or methionine (when the R³s are (CH₂)₂SCH₃), and combinations thereof. In yet another alternative embodiment, all of the various α-amino acids contained in the polymers used in making the invention OEG-based polymer compositions are biological α-amino acids, as described herein.

The polymer compositions can be formulated to provide a variety of properties. In one embodiment, particles of the invention PEEA, PEEUR and PEEU polymers are sized to agglomerate in vivo forming a time-release polymer depot when injected in vivo for local delivery of bioactive agents dispersed therein to surrounding tissue/cells.

Accordingly, in yet another embodiment, the invention provides methods for delivering one or more bioactive agents to a local site in the body in a subject. In this embodiment, the invention methods involve injecting into an in vivo site in the body of the subject an invention OEG-based polymer composition that has been formulated as polymer particles with at least one bioactive agent dispersed therein. The injected particles agglomerate to form a polymer depot of particles of increased size and the agglomeration will slowly release the individual particles, which will biodegrade by enzymatic action as described herein to release the dispersed bioactive agent(s). After biodegradation, each polymer molecule also will release into the circulation from 15 to 300 oligo-ethylene glycol (OEG) molecules having a molecular weight of 44 up to, but not including 400 Da. As described herein, the number of oligo-ethylene glycol (OEG) molecules having a molecular weight of 400 Da or less released by biodegradation of any particular polymer will depend upon selection of the diols and di-acids used in fabrication of the polymer.

A dispersion of the OEG-based PEEA, PEEUR or PEEU particles can be injected parenterally, for example subcutaneously, intramuscularly, or into an interior body site, such as an organ. Polymer particles of sizes capable of passing through pharmaceutical syringe needles ranging in size from about 19 to about 27 Gauge, for example those having an average diameter in the range from about 1 μm to about 200 μm, can be injected into an interior body site, and will agglomerate to form particles of increased size that form a depot to dispense the dispersed bioactive agent(s) locally. In other embodiments, the biodegradable polymer particles act as a carrier for the bioactive agent into the circulation for targeted and timed release systemically. Invention polymer particles in the size range of about 10 nm to about 500 nm will enter directly into the circulation for such purposes.

The biodegradable polymers used in the invention OEG-based polymer composition can be designed to tailor the rate of biodegradation of the polymer to result in controlled delivery of the bioactive agent over a period of time. For instance, typically, a thin disc of the invention composition will biodegrade by surface erosion in enzymatic solution (e.g., such as is found in vivo) so as to undergo a weight loss of about 81% to 56% weight loss within 24 hrs.

The present invention utilizes biodegradable polymer particle-mediated delivery techniques to deliver a wide variety of bioactive agents in treatment of a wide variety of diseases and disease symptoms at a rate that can be engineered by selection of the polymer and particle size. Although certain of the individual components of the polymer composition and methods described herein were known, it was unexpected and surprising that such combinations would enable the practitioner to select a biodegradable polymer, bioactive agent and particle size to control the time interval during which the bioactive agent is released beyond levels of control heretofore achieved.

Polymers suitable for use in the practice of the invention described by structural formula (I and IV-VIII) bear functionalities that allow facile covalent attachment of the bioactive agent(s) or covering molecule(s) to the polymer. For example, a polymer bearing carboxyl groups can readily react with an amino moiety, thereby covalently bonding a peptide to the polymer via the resulting amide group. As will be described herein, the biodegradable polymer and the bioactive agent may contain numerous complementary functional groups that can be used to covalently attach the bioactive agent to the biodegradable polymer. For example, PEEURs synthesized by the polycondensation of active bis-carbonates (compounds 4a-c in Example 2) with oligo-ethylene glycol (OEG) based di-p-toluensulfonic acid salts (compound 2a-e below and in Example 2) can be useful as biodegradable analogs of PEGs for various chemical and biochemical applications:

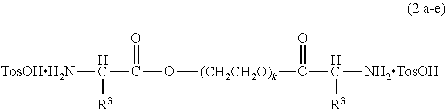

The monomers 2a-e can be synthesized by direct condensation of α-amino acids with OEG as are monomers described in the Examples below. Alternatively, using methods of peptide chemistry well known to those of skill in the art, α-amino acids of various hydrophobicities can be used for synthesizing the monomers 2a-e. The latter method will allow regulation of the hydrophilic/hydrophobic balance of the invention biodegradable OEG-based polymers. Hence, the properties and biological applications of the invention polymers and compositions can vary in a wide range.

For example, all the heterolinkages in the backbones of PEEURs—ether and urethane bonds—are hydrophilic, thus enhancing water solubility of these polymers. By judicious selection of the lateral R³ substituents in the α-amino acids that link together the OEG segments, the hydrophilic/hydrophobic balance of the invention OEG-containing polymers can be regulated using principles well known in the art and as described herein.

The invention PEEAs, PEEURs and PEEUs, like other polymers obtained via solution active polycondensation, contain two terminal groups that can be used to functionalize these polymers for their subsequent chemical/biochemical applications using methods known in the art and as described herein. Moreover, by interaction with mono-ethanol amine, the terminal active carbonate groups of the polymers can be transformed into oxy-ethyl urethane groups to enhance hydrophilicity of the polymers.

Alternatively still, terminal OH groups can be used for subsequent transformations, e.g. to attach acrylates. In a similar way, SH-terminated PEEURs can be synthesized by interaction with mercapto-amines. The SH-terminated PEEURs can be attached under mild conditions to polymers containing active double bond moieties, e.g. to fumaric acid based unsaturated PEAs, or can be used for subsequent transformations, as described below.

The invention PEEURs also can be used for chemical/photo-chemical grafting to other unsaturated polymers, e.g. to fumaric acid-based unsaturated PEAs, and the like, to render them hydrophilic or water soluble.

PEEAs, PEEURs and PEEUs terminated with unsaturated maleimide cycles are of interest for the attachment of the polymers used in the invention compositions to HS-containing molecules, for example to proteins, enzymes, peptides, and the like, and can be synthesized in several ways. For example, maleimide terminated polymers can be synthesized by interaction of terminal amino groups with excess of N,N′-alkylene-bis-maleimides or with active diester, e.g. N-maleimido-β-alanine.

SH-terminated polymers (formed as described above) also can be used to synthesize maleimide terminated PEEAs, PEEURs and PEEUs by interaction of terminal amino groups with excess of N,N′-alkylene-bis-maleimides.

The terminal active carbonate groups can be transformed into maleimide terminus according to the following scheme:

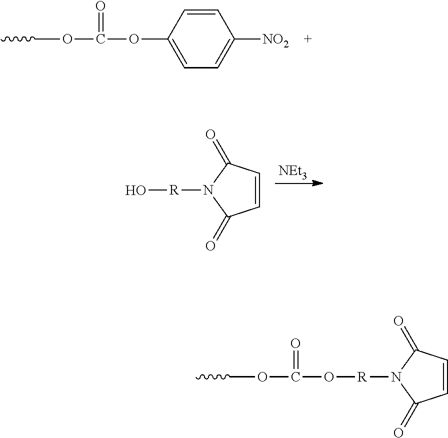

The polymer in the invention OEG-based polymer composition plays an active role in the treatment processes at the site of local injection by holding the bioactive agent at the site of injection for a period of time sufficient to allow the individual's endogenous processes to interact with the bioactive agent, while releasing the particles or polymer molecules containing such agents during biodegradation of the polymer. The fragile bioactive agent is protected by the more slowly biodegrading polymer to increase half-life and persistence of the bioactive agent(s).

In addition, the polymers disclosed herein (e.g., those having structural formulas (I and IV-VIII), upon enzymatic degradation, provide α-amino acids while the other breakdown products are either OEGs or, if not, are diols or di-acids that can be metabolized in the way that fatty acids and sugars are metabolized. Uptake of the polymer is safe: studies have shown that the subject can metabolize and clear the polymer degradation products. These polymers and the invention OEG-based polymer compositions are, therefore, substantially non-inflammatory to the subject both at the site of injection and systemically, apart from any trauma caused by injection itself.

In the PEEA, PEEUR and PEEU polymers useful in practicing the invention, multiple different α-amino acids can be employed in a single polymer molecule. The polymers may comprise at least two different amino acids per repeat unit and a single polymer molecule may contain, multiple different α-amino acids in the polymer molecule, depending upon the size of the molecule.

The polymers described herein may also be used in a block co-polymer. In yet another embodiment, the polymer is used as one block in di- or tri-block copolymers, which, for example, can be used to make micelles, as described below.

The invention OEG-based polymer compositions and methods are members of the larger family of polyester amides (PEAs), polyester urethanes (PEURs) and polyester ureas (PEUs), many of which have built-in functional groups on side chains, and these built-in functional groups can react with other chemicals and lead to the incorporation of additional functional groups to expand the functionality of the polymers further. Similarly, the OEG-based (i.e., PEEA, PEEUR or PEEU) polymers used in the invention methods are ready for reaction with other chemicals having a hydrophilic structure to increase water solubility and with bioactive agents and covering molecules, without the necessity of prior modification.

In addition, the OEG-based polymers used in the invention OEG-based polymer compositions display minimal hydrolytic degradation when tested in a saline (PBS) medium, but in an enzymatic solution, such as lipase, chymotrypsin or CT, a uniform surface erosive behavior has been observed that forms a substantially zero-order release profile, as described herein.

Suitable protecting groups for use in the PEEA, PEEUR and PEEU polymers include t-butyl or another as is known in the art. Suitable 1,4:3,6-dianhydrohexitols of general formula (III) include those derived from sugar alcohols, such as D-glucitol, D-mannitol, or L-iditol. Dianhydrosorbitol is the presently preferred bicyclic fragment of a 1,4:3,6-dianhydrohexitol for use in the PEEA, PEEUR and PEEU polymers used in fabrication of the invention OEG-based polymer compositions.

The term “Oligo-ethylene glycol (OEG)” as used herein means an oligomer or polymer of ethylene oxide having the chemical structure HO—(CH₂—CH₂—O)₁₋₉—H. The terms “oligo-ethylene glycol-containing (OEG-containing)” moiety and “oligo-ethylene glycol-based (OEG-based)” moiety are used herein to refer to polymer segments that release an OEG molecule upon enzymatic biodegradation of the polymer, which OEG may be either monodisperse or, more commonly, polydisperse with a polydispersity index in the range from about 1.05 up to 2.0. A polydisperse OEG molecule as described herein is characterized statistically by its weight average molecular weight)(Mw) and its number average molecular weight (Mn), the ratio of which is called the polydispersity index (Mw/Mn).

The molecular weights and polydispersities herein are determined by gel permeation chromatography (GPC) using polystyrene standards. More particularly, number and weight average molecular weights (Mₙ and M_{w}) are determined, for example, using a Model 510 gel permeation chromatography (Water Associates, Inc., Milford, Mass.) equipped with a high-pressure liquid chromatographic pump, a Waters 486 UV detector and a Waters 2410 differential refractive index detector. Tetrahydrofuran (THF), N,N-dimethylformamide (DMF) or N,N-dimethylacetamide (DMAc) is used as the eluent (1.0 mL/min). Polystyrene or poly(methyl methacrylate) standards having narrow molecular weight distribution were used for calibration.

Methods for making polymers containing a α-amino acid in the general formula are well known in the art. For example, for the embodiment of the polymer of structural formula (I) wherein R⁴ is incorporated into an α-amino acid, for polymer synthesis the α-amino acid with pendant R³ can be converted through esterification into a bis-α,ω-diamine. For example, the α-amino acid containing pendant R³ can be condensed with at least one oligo-ethylene glycol-based diol (OEG diol). As a result, OEG-containing bis-α-aminoacyl diester monomers with reactive α,ω-amino groups are formed. Then, the bis-α,ω-diamine is entered into a polycondensation reaction with a di-acid, such as adipic, sebacic or fumaric acid, or bis-activated esters, or bis-acyl chlorides, to obtain the final polymer having an OEG-containing moiety, as well as ester, ether and amide bonds (PEEA). Alternatively, for example, for polymers of structure (I), instead of the di-acid, an activated di-acid derivative, e.g., bis-para-nitrophenyl diester, can be used as an activated di-acid. Additionally, a bis-di-carbonate, such as bis(p-nitrophenyl) oligoether-containing dicarbonate, can be used as the activated species to obtain polymers containing a residue of a diol that contains two to nine ethylene oxide functionalities (i.e., an OEG-containing moiety). In the case of PEEUR polymers, a final polymer is obtained having an OEG-containing moiety, as well as ester, ether and urethane bonds.

OEG-containing di-acid-type compounds useful for active polycondensation according to the present invention are α,ω-alkylene dicarboxylates of formula (III) composed of short aliphatic non toxic di-acids and OEGs as diols.

These molecules inherently contain at least one ester group, which easily can be cleaved by biotic (enzymatic) and abiotic hydrolysis. The invention PEEA, PEEUR and PEEU polymer compositions possess increased hydrophilicity and an increased number of ester groups in the backbone chain per unit as compared with previously known PEA, PEUR and PEU polymers, which ester groups in some cases confer more rapid biodegradability than polymers composed of aliphatic diols and di-acids with alkylene chains.

As illustration of the first stage of synthesis, a variety of OEG-containing new diester-di-acids (or alkylene-dicarboxylates) of structural formula (III) can be fabricated by interaction of diols with cyclic aliphatic five or six-member anhydrides, such as maleic, succinic and glutaric anhydrides. The general scheme of alkylene-dicarboxylate synthesis is depicted in reaction Scheme I below:

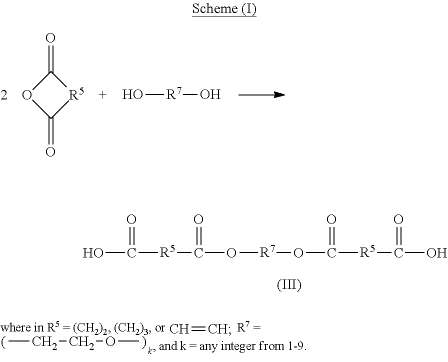

As illustration of the second stage of synthesis, various active di-(p-nitrophenyl) esters of alkylene-dicarboxylates of structural Formula IX have been fabricated. The reaction is accomplished by interaction of di-acids formed in the first stage (Formula II) with p-nitrophenol in the presence of different condensing agents.

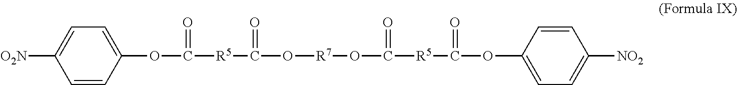

wherein R⁵ can be selected, for example, from (CH₂)₂, (CH₂)₃, CH═CH; and R⁷ can be selected from R⁷═(—CH₂—CH₂—O—)ₖ, where k=any integer from 1-9.

The unsaturated PEEAs can be prepared by solution polycondensation of either (1) di-p-toluene sulfonic acid salt of bis(α-amino acid) di-ester of unsaturated diol and di-p-nitrophenyl ester of saturated dicarboxylic acid or (2) di-p-toluene sulfonic acid salt of bis(α-amino acid) diester of saturated diol and di-nitrophenyl ester of unsaturated dicarboxylic acid or (3) di-p-toluene sulfonic acid salt of bis(α-amino acid) diester of unsaturated diol and di-nitrophenyl ester of unsaturated dicarboxylic acid. In the present invention at least one, and preferably all, of the diols and/or di-acids used in fabrication contains from one to eight ether functionalities.

Salts of p-toluene sulfonic acids are known for use in synthesizing polymers containing amino acid residues. The aryl sulfonic acid salts are used instead of the free base because the aryl sulfonic salts of bis (α-amino acid) diesters are easily purified through recrystallization and render the amino groups as unreactive ammonium tosylates throughout workup. In the polycondensation reaction, the nucleophilic amino group is readily revealed through the addition of an organic base, such as triethylamine, so the polymer product is obtained in high yield.

For polymers of structural formula (I), for example, the di-p-nitrophenyl esters of unsaturated dicarboxylic acid can be synthesized from p-nitrophenyl and unsaturated dicarboxylic acid chloride, e.g., by dissolving triethylamine and p-nitrophenol in acetone and adding unsaturated dicarboxylic acid chloride dropwise with stirring at −78° C. and pouring into water to precipitate product. Suitable acid chlorides included fumaric, maleic, mesaconic, citraconic, glutaconic, itaconic, ethenyl-butane dioic and 2-propenyl-butanedioic acid chlorides. For polymers of structure (V) and (VI), bis-p-nitrophenyl dicarbonates of saturated or unsaturated diols are used as the activated monomer. Dicarbonate monomers of general structure (X) are employed for polymers of structural formula (V) and (VI), wherein each R¹⁰ is independently (C₆-C₁₀) aryl optionally substituted with one or more nitro, cyano, halo, trifluoromethyl, or trifluoromethoxy; and R⁶ can be as described above.

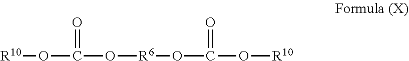

The di-aryl sulfonic acid salts of diesters of α-amino acid and unsaturated diol can be prepared by admixing α-amino acid, e.g., p-aryl sulfonic acid monohydrate and saturated or unsaturated diol in toluene, heating to reflux temperature, until water evolution is minimal, then cooling. The unsaturated diols that do not contain ether functionalities include, for example, 2-butene-1,3-diol and 1,18-octadec-9-en-diol.

Saturated di-p-nitrophenyl esters of dicarboxylic acid and saturated di-p-toluene sulfonic acid salts of bis-α-amino acid esters can be prepared as described in U.S. Pat. No. 6,503,538 B1. Synthesis of the unsaturated poly(ester amide)s (UPEAs) useful as biodegradable polymers are known in the art (See e.g., U.S. Pat. Nos. 5,516,881; 6,476,204; 6,503,538). Synthesis of unsaturated PEEAs of the structural formula (I) is as disclosed above and as described in Example 1 herein.

In unsaturated compounds having either structural formula (I) or (IV), the following hold. An amino substituted aminoxyl (N-oxide) radical bearing group, e.g., 4-amino TEMPO, can be attached using carbonyldiimidazol, or suitable carbodiimide, as a condensing agent. Bioactive agents, as described herein, can be attached via the double bond functionality. Hydrophilicity can be imparted by bonding to poly(ethylene glycol) diacrylate.

The biodegradable PEEA, PEEUR and PEEU polymers can contain from one to multiple different α-amino acids per polymer molecule and preferably have weight average molecular weights ranging from 10,000 to 125,000 g/mol; these polymers and copolymers typically have intrinsic viscosities at 25° C., determined by standard viscosimetric methods, ranging from 0.3 to 3.0, for example, ranging from 0.5 to 1.5.

In unsaturated compounds having structural formula (VII) for PEEUs the following hold: An amino substituted aminoxyl (N-oxide) radical bearing group e.g., 4-amino TEMPO, can be attached using carbonyldiimidazole, or suitable carbodiimide, as a condensing agent. Additional bioactive agents, and the like, as described herein, optionally can be attached via the double bond functionality.

For example, the invention high Mw PEEUs having structural formula (VII) can be prepared inter-facially by using phosgene as a bis-electrophilic monomer in a chlorofounfwater system, as shown in the reaction scheme (II) below:

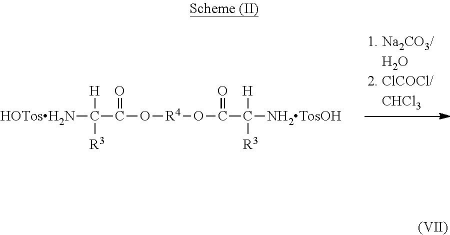

Synthesis of copoly(ester ureas) (PEEUs) containing L-Lysine esters and having structural formula (VIII) can be carried out by a similar scheme (III):

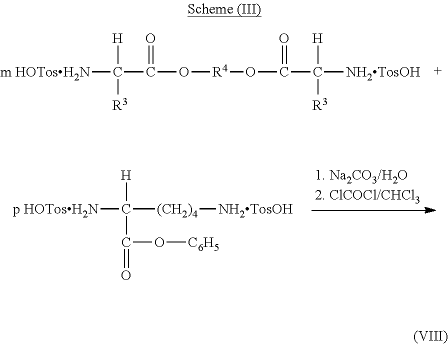

A 20% solution of phosgene (highly toxic) in toluene, (Fluka Chemie, GMBH, Buchs, Switzerland), can be replaced either by diphosgene (trichloromethylchloroformate) or triphosgene (bis(trichloromethyl)carbonate). Less toxic carbonyldiimidazole can be also used as a bis-electrophilic monomer instead of phosgene, di-phosgene, or tri-phosgene.

It is necessary to use cooled solutions of monomers to obtain PEEUs of high Mw. For example, to a suspension of di-p-toluenesulfonic acid salt of bis(α-amino acid)-α,ω-alkylene diester in 150 mL of water, anhydrous sodium carbonate is added, stirred at room temperature for about 30 minutes and cooled to about 2-0° C., forming a first solution. In parallel, a second solution of phosgene in chloroform is cooled to about 15-10° C. The first solution is placed into a reactor for interfacial polycondensation and the second solution is quickly added at once and stirred briskly for about 15 min. Then a chloroform layer can be separated, dried over anhydrous Na₂SO₄, and filtered. The obtained solution can be stored for further use.

PEU polymers fabricated have been obtained as solutions in chloroform and these solutions are stable during storage. However, some polymers, for example, 1-Phe-4, become insoluble in chloroform after separation. To overcome this problem, PEEU polymers can be separated from chloroform solution by casting onto a smooth hydrophobic surface and allowing chloroform to evaporate to dryness. No further purification of obtained PEEUs is needed. General procedures to preparation of PEUs are described in published U.S application US2007/0128250-A1.

Polymers useful in the invention OEG-based polymer compositions, such as PEEA, PEEUR and PEEU polymers, biodegrade by enzymatic action at the surface. Therefore, the polymers, for example particles thereof, administer the bioactive agent to the subject at a controlled release rate, for which the kinetics have been observed to be close to zero order. Additionally, since PEEA, PEEUR and PEEU polymers break down in vivo via hydrolytic enzymes without production of adverse side products, the invention OEG-based polymer compositions are substantially non-inflammatory. Even the OEGs incorporated therein are of such low Mw that they can be cleared from the body once liberated from the polymer after biodegradation.

As used herein “dispersed” means at least one bioactive agent as disclosed herein is dispersed, mixed, dissolved, homogenized, and/or covalently bound (“dispersed”) in a polymer particle, for example attached to the surface of the particle.

While the bioactive agents can be dispersed within the polymer matrix without chemical linkage to the polymer carrier, it is also contemplated that the bioactive agent or a covering molecule can be covalently bound to the biodegradable polymers via a wide variety of suitable functional groups. For example, when the biodegradable polymer is a polyester, the carboxyl group chain end can be used to react with a complimentary moiety on the bioactive agent or covering molecule, such as hydroxy, amino, thio, and the like. A wide variety of suitable reagents and reaction conditions are disclosed, e.g., in March's Advanced Organic Chemistry, Reactions, Mechanisms, and Structure, Fifth Edition, (2001); and Comprehensive Organic Transformations, Second Edition, Larock (1999).

In other embodiments, a bioactive agent can be linked to the PEEA, PEEUR or PEEU polymers described herein through an amide, ester, ether, amino, ketone, thioether, sulfmyl, sulfonyl, disulfide linkage. Such a linkage can be formed from suitably functionalized starting materials using synthetic procedures that are known in the art.

For example, in one embodiment a polymer can be linked to the bioactive agent via an end or pendent carboxyl group (e.g., COOH) of the polymer. For example, a compound of structures, IV, VI and VIII can react with an amino functional group or a hydroxyl functional group of a bioactive agent to provide a biodegradable polymer having the bioactive agent attached via an amide linkage or carboxylic ester linkage, respectively. In another embodiment, the carboxyl group of the polymer can be benzylated or transformed into an acyl halide, acyl anhydride/“mixed” anhydride, or active ester. In other embodiments, the free —NH₂ ends of the polymer molecule can be acylated to assure that the bioactive agent will attach only via a carboxyl group of the polymer and not to the free ends of the polymer.

Water soluble covering molecule(s), such as poly(ethylene glycol) (PEG); phosphoryl choline (PC); glycosaminoglycans including heparin; polysaccharides including polysialic acid; poly(ionizable or polar amino acids) including polyserine, polyglutamic acid, polyaspartic acid, polylysine and polyarginine; chitosan and alginate, as described herein; and targeting molecules, such as antibodies, antigens and ligands, can also be conjugated to the polymer on the exterior of the particles after production of the particles to block active sites not occupied by the bioactive agent or to target delivery of the particles to a specific body site as is known in the art. The Mws of OEG molecules formed upon biodegradation of a single particle, including covering molecules, can be substantially any Mw in the range from about 44 (monoethyleneglycol) up to, but not including 400 Da, so that the Mws of the various PEG molecules attached to the particle can be varied.

Alternatively, the bioactive agent or covering molecule can be attached to the polymer via a linker molecule. Indeed, to improve surface hydrophobicity of the biodegradable polymer, to improve accessibility of the biodegradable polymer towards enzyme activation, and to improve the release profile of the biodegradable polymer, a linker may be utilized to indirectly attach the bioactive agent to the biodegradable polymer. In certain embodiments, the linker compounds include poly(ethylene glycol) having a Mw of about 44 up to 400 Da, preferably 200 up to 400; amino acids, such as serine; polypeptides with repeat number from 1 to 100; and any other suitable low molecular weight polymers. The linker typically separates the bioactive agent from the polymer by about 5 angstroms up to about 200 angstroms.

In still further embodiments, the linker is a divalent radical of formula W-A-Q, wherein A is (C₁-C₂₄) alkyl, (C₂-C₂₄) alkenyl, (C₂-C₂₄) alkynyl, (C₃-C₈) cycloalkyl, or (C₆-C₁₀) aryl, and W and Q are each independently —N(R)C(═O)—, —C(═)O, —O—, —S—, —S(O), —S(O)₂—, —S—S—, —N(R)—, —C(═O)—, wherein each R is independently H or (C₁-C₆) alkyl.

As used to describe the above linkers, the term “alkyl” refers to a straight or branched chain hydrocarbon group including methyl, ethyl, n-propyl, isopropyl, n-butyl, isobutyl, tert-butyl, n-hexyl, and the like.

As used herein to describe the above linkers, “alkenyl” refers to straight or branched chain hydrocarbyl groups having one or more carbon-carbon double bonds.

As used herein to describe the above linkers, “alkynyl” refers to straight or branched chain hydrocarbyl groups having at least one carbon-carbon triple bond.

As used herein to describe the above linkers, “aryl” refers to aromatic groups having in the range of 6 up to 14 carbon atoms.

In certain embodiments, the linker may be a polypeptide having from about 2 up to about 25 amino acids. Suitable peptides contemplated for use include poly-L-glycine, poly-L-lysine, poly-L-glutamic acid, poly-L-aspartic acid, poly-L-histidine, poly-L-ornithine, poly-L-serine, poly-L-threonine, poly-L-tyrosine, poly-L-leucine, poly-L-lysine-L-phenylalanine, poly-L-arginine, poly-L-lysine-L-tyrosine, and the like.

In one embodiment, the bioactive agent covalently crosslinks the polymer, i.e. the bioactive agent is bound to more than one polymer molecule. This covalent crosslinking can be done with or without additional polymer-bioactive agent linker.

The bioactive agent molecule can also be incorporated into an intramolecular bridge by covalent attachment between two polymer molecules.

A linear polymer polypeptide conjugate is made by protecting the potential nucleophiles on the polypeptide backbone and leaving only one reactive group to be bound to the polymer or polymer linker construct. Deprotection is performed according to methods well known in the art for deprotection of peptides (Boc and Fmoc chemistry for example).

In one embodiment of the present invention, a polypeptide bioactive agent is presented as retro-inverso or partial retro-inverso peptide.

In other embodiments the bioactive agent is mixed with a photocrosslinkable version of the polymer in a matrix, and after crosslinking the material is dispersed (ground) to an average diameter in the range from about 0.1 to about 10 μm.

The linker can be attached first to the polymer or to the bioactive agent or covering molecule. During synthesis, the linker can be either in unprotected form or protected form, using a variety of protecting groups well known to those skilled in the art. In the case of a protected linker, the unprotected end of the linker can first be attached to the polymer or the bioactive agent or covering molecule. The protecting group can then be de-protected using Pd/H₂ hydrogenolysis, mild acid or base hydrolysis, or any other common de-protection method that is known in the art. The de-protected linker can then be attached to the bioactive agent or covering molecule, or to the polymer

An exemplary synthesis of a biodegradable polymer according to the invention (wherein the molecule to be attached is an aminoxyl) is set forth as follows.

A polyester can be reacted with an amino-substituted aminoxyl (N-oxide) radical bearing group, e.g., 4-amino-2,2,6,6-tetramethylpiperidine-1-oxy, in the presence of N,N-carbonyldiimidazole to replace the hydroxyl moiety in the carboxyl group at the chain end of the polyester with an amino-substituted aminoxyl-(N-oxide) radical bearing group, so that the amino moiety covalently bonds to the carbon of the carbonyl residue of the carboxyl group to form an amide bond. The N,N′-carbonyl diimidazole or suitable carbodiimide converts the hydroxyl moiety in the carboxyl group at the chain end of the polyester into an intermediate product moiety which will react with the aminoxyl, e.g., 4-amino-2,2,6,6-tetramethylpiperidine-1-oxy. The aminoxyl reactant is typically used in a mole ratio of reactant to polyester ranging from 1:1 to 100:1. The mole ratio of N,N′-carbonyl diimidazole to aminoxyl is preferably about 1:1.

A typical reaction is as follows. A polyester is dissolved in a reaction solvent and reaction is readily carried out at the temperature utilized for the dissolving. The reaction solvent may be any in which the polyester will dissolve. When the polyester is a polyglycolic acid or a poly(glycolide-L-lactide) (having a monomer mole ratio of glycolic acid to L-lactic acid greater than 50:50), highly refined (99.9+% pure) dimethyl sulfoxide at 115° C. to 130° C. or DMSO at room temperature suitably dissolves the polyester. When the polyester is a poly-L-lactic acid, a poly-DL-lactic acid or a poly(glycolide-L-lactide) (having a monomer mole ratio of glycolic acid to L-lactic acid 50:50 or less than 50:50), tetrahydrofuran, dichloromethane (DCM) and chloroform at room temperature to 40-50° C. suitably dissolve the polyester.

### Polymer—Bioactive Agent Linkage

In one embodiment, the polymers used to make the invention OEG-based polymer compositions as described herein have one or more bioactive agent directly linked to the polymer. The residues of the polymer can be linked to the residues of the one or more bioactive agents. For example, one residue of the polymer can be directly linked to one residue of the bioactive agent. The polymer and the bioactive agent can each have one open valence. Alternatively, more than one bioactive agent, multiple bioactive agents, or a mixture of bioactive agents having different therapeutic or palliative activity can be directly linked to the polymer. However, since the residue of each bioactive agent can be linked to a corresponding residue of the polymer, the number of residues of the one or more bioactive agents can correspond to the number of open valences on the residue of the polymer.

As used herein, a “residue of a polymer” refers to a radical of a polymer having one or more open valences. Any synthetically feasible atom, atoms, or functional group of the polymer (e.g., on the polymer backbone or pendant group) of the present invention can be removed to provide the open valence, provided bioactivity is substantially retained when the radical is attached to a residue of a bioactive agent. Additionally, any synthetically feasible functional group (e.g., carboxyl) can be created on the polymer (e.g., on the polymer backbone or pendant group) to provide the open valence, provided bioactivity is substantially retained when the radical is attached to a residue of a bioactive agent. Based on the linkage that is desired, those skilled in the art can select suitably functionalized starting materials that can be derived from the polymer of the present invention using procedures that are known in the art.

As used herein, a “residue of a compound of structural formula (*)” refers to a radical of a compound of polymer formulas (I) and (IV-VIII) as described herein having one or more open valences. Any synthetically feasible atom, atoms, or functional group of the compound (e.g., on the polymer backbone or pendant group) can be removed to provide the open valence, provided bioactivity is substantially retained when the radical is attached to a residue of a bioactive agent. Additionally, any synthetically feasible functional group (e.g., carboxyl) can be created on the compound of formulas (I) and (IV-VIII) (e.g., on the polymer backbone or pendant group) to provide the open valance, provided bioactivity is substantially retained when the radical is attached to a residue of a bioactive agent. Based on the linkage that is desired, those skilled in the art can select suitably functionalized starting materials that can be derived from the compound of formulas (I) and (IV-VIII) using procedures that are known in the art.

For example, the residue of a bioactive agent can be linked to the residue of a compound of structural formula (I) or (IV) through an amide (e.g., —N(R)C(═O)— or —C(═O)N(R)—), ester (e.g., —OC(═O)— or —C(═O)O—), ether (e.g., —O—), amino (e.g., —N(R)—), ketone (e.g., —C(═O)—), thioether (e.g., —S—), sulfinyl (e.g., —S(O)—), sulfonyl (e.g., —S(O)₂—), disulfide (e.g., —S—S—), or a direct (e.g., C—C bond) linkage, wherein each R is independently H or (C₁-C₆) alkyl. Such a linkage can be formed from suitably functionalized starting materials using synthetic procedures that are known in the art. Based on the linkage that is desired, those skilled in the art can select suitably functional starting material that can be derived from a residue of a compound of structural formula (I) or (IV) and from a given residue of a bioactive agent or adjuvant using procedures that are known in the art. The residue of the bioactive agent or adjuvant can be linked to any synthetically feasible position on the residue of a compound of structural formula (I) or (IV). Additionally, the invention also provides compounds having more than one residue of a bioactive agent or adjuvant bioactive agent directly linked to a compound of structural formula (I) or (IV).

The number of bioactive agents that can be linked to the polymer molecule can typically depend upon the molecular weight of the polymer. For example, for a compound of structural formula (I), wherein n is about 5 to about 150, preferably about 5 to about 70, up to about 150 bioactive agent molecules (i.e., residues thereof) can be directly linked to the polymer (i.e., residue thereof) by reacting the bioactive agent with side groups of the polymer. In unsaturated polymers, the bioactive agents can also be reacted with double bonds in the polymer.

PEEA, PEEUR and PEEU polymers described herein absorb water, (5 to 25% w/w water up-take, on polymer film) allowing hydrophilic molecules to readily diffuse therethrough. This characteristic makes these polymers suitable for use as an over coating on particles to control release rate. Water absorption also enhances biocompatibility of the polymers and the polymer composition based on such polymers. In addition, due to the hydrophilic properties of the PEEA, PEEUR and PEEU polymers, when delivered in vivo the particles become sticky and agglomerate, particularly at in vivo temperatures. Thus the polymer particles spontaneously form polymer depots when injected subcutaneously or intramuscularly for local delivery, such as by subcutaneous needle or needle-less injection. Particles with average diameter range from about 1 micron to about 100 microns, which size will not circulate efficiently within the body, are suitable for forming such polymer depots in vivo. Alternatively, for oral administration the GI tract can tolerate much larger particles, for example micro particles of about 1 micron up to about 1000 microns average diameter.

Particles suitable for use in the invention OEG-based polymer compositions can be made using immiscible solvent techniques. Generally, these methods entail the preparation of an emulsion of two immiscible liquids. A single emulsion method can be used to make polymer particles that incorporate at least one hydrophobic bioactive agent. In the single emulsion method, bioactive agents to be incorporated into the particles are mixed with polymer in solvent first, and then emulsified in water solution with a surface stabilizer, such as a surfactant. In this way, polymer particles with hydrophobic bioactive agent conjugates are formed and suspended in the water solution, in which hydrophobic conjugates in the particles will be stable without significant elution into the aqueous solution, but such molecules will elute into body tissue, such as muscle tissue.

Most biologics as the term is used herein, including polypeptides, proteins, DNA, cells and the like, are hydrophilic. A double emulsion method can be used to make polymer particles with interior aqueous phase and hydrophilic bioactive agents dispersed within. In the double emulsion method, aqueous phase or hydrophilic bioactive agents dissolved in water are emulsified in polymer lipophilic solution first to form a primary emulsion, and then the primary emulsion is put into water to emulsify again to form a second emulsion, in which particles are formed having a continuous polymer phase and aqueous bioactive agent(s) in the dispersed phase. Surfactant and additive can be used in both emulsifications to prevent particle aggregation. Chloroform or DCM, which are not miscible in water, are used as solvents for PEA and PEUR polymers, but later in the preparation the solvent is removed, using methods known in the art.

For certain bioactive agents with low water solubility, however, these two emulsion methods have limitations. In this context, “low water solubility” means a bioactive agent that is less hydrophobic than truly lipophilic drugs, such as Taxol, but which are less hydrophilic than truly water-soluble drugs, such as many biologics. These types of intermediate compounds are too hydrophilic for high loading and stable matrixing into single emulsion particles, yet are too hydrophobic for high loading and stability within double emulsions. In such cases, a polymer layer is coated onto particles made of polymer and drugs with low water solubility, by a triple emulsion process. This method provides relatively low drug loading (˜10% w/w), but provides structure stability and controlled drug release rate.

In the triple emulsion process, the first emulsion is made by mixing the bioactive agents into polymer solution and then emulsifying the mixture in aqueous solution with surfactant or lipid, such as di-(hexadecanoyl)phosphatidylcholine (DHPC; a short-chain derivative of a natural lipid). In this way, particles containing the active agents are formed and suspended in water to form the first emulsion. The second emulsion is formed by putting the first emulsion into a polymer solution, and emulsifying the mixture, so that water drops with the polymer/drug particles inside are formed within the polymer solution. Water and surfactant or lipid will separate the particles and dissolve the particles in the polymer solution. The third emulsion is then formed by putting the second emulsion into water with surfactant or lipid, and emulsifying the mixture to form the final particles in water. The resulting particle structure will have one or more particles made with polymer plus bioactive agent at the center, surrounded by water and surface stabilizer, such as surfactant or lipid, and covered with a pure polymer shell. Surface stabilizer and water will prevent solvent in the polymer coating from contacting the particles inside the coating and dissolving them.

To increase loading of bioactive agents by the triple emulsion method, active agents with low water solubility can be coated with surface stabilizer in the first emulsion, without polymer coating and without dissolving the bioactive agent in water. In this first emulsion, water, surface stabilizer and active agent have similar volume or in the volume ratio range of (1 to 3):(0.2 to about 2):1, respectively. In this case, water is used, not for dissolving the active agent, but rather for protecting the bioactive agent with help of surface stabilizer. Then the double and triple emulsions are prepared as described above. This method can provide up to 50% drug loading.

Alternatively or additionally, in the single, double or triple emulsion methods described above, a bioactive agent can be conjugated to the polymer molecule as described herein prior to using the polymers to make the particles. Alternatively still, a bioactive agent can be conjugated to the polymer on the exterior of the particles described herein after production of the particles.

Many emulsification techniques will work in making the emulsions described above. However, the presently preferred method of making the emulsion is by using a solvent that is not miscible in water. For example, in the single emulsion method, the emulsifying procedure consists of dissolving polymer with the solvent, mixing with bioactive agent molecule(s), putting into water, and then stirring with a mixer and/or ultra-sonicator. Particle size can be controlled by controlling stir speed and/or the concentration of polymer, bioactive agent(s), and surface stabilizer. Coating thickness can be controlled by adjusting the ratio of the second to the third emulsion.

Suitable emulsion stabilizers may include nonionic surface active agents, such as mannide monooleate, dextran 70,000, polyoxyethylene ethers, polyglycol ethers, and the like, all readily commercially available from, e.g., Sigma Chemical Co., St. Louis, Mo. The surface active agent will be present at a concentration of about 0.3% to about 10%, preferably about 0.5% to about 8%, and more preferably about 1% to about 5%.

Rate of release of the at least one bioactive agent from the invention compositions can be controlled by adjusting the coating thickness, particle size, structure, and density of the coating. Density of the coating can be adjusted by adjusting loading of the bioactive agent conjugated to the coating. For example, when the coating contains no bioactive agent, the polymer coating is densest, and a bioactive agent from the interior of the particle elutes through the coating most slowly. By contrast, when a bioactive agent is loaded into (i.e. is mixed or “matrixed” with), or alternatively is conjugated to, polymer in the coating, the coating becomes porous once the bioactive agent has become free of polymer and has eluted out, starting from the outer surface of the coating. Thereby, a bioactive agent at the center of the particle can elute at an increased rate. The higher the bioactive agent loading in the coating, the lower the density of the coating layer and the higher the elution rate. The loading of bioactive agent in the coating can be lower or higher than that in the interior of the particles beneath the exterior coating. Release rate of bioactive agent(s) from the particles can also be controlled by mixing particles with different release rates prepared as described above.

A detailed description of methods of making double and triple emulsion polymers may be found in Pierre Autant et al, U.S. Pat. No. 6,022,562; Iosif Daniel Rosca et al., Microparticle formation and its mechanism in single and double emulsion solvent evaporation, Journal of Controlled Release (2004) 99:271-280; L. Mu and A Feng, A novel controlled release formulation for the anticancer drug paclitaxel (Taxol): PLGA nanoparticles containing vitamin E TPGS, J Control. Release (2003) 86:33-48; Somatosin containing biodegradable microspheres prepared by a modified solvent evaporation method based on W/O/W-multiple emulsions, Int. J Pharm. 126 (1995) 129-138 and F. Gabor et al., Ketoprofenpoly(d,l-lactic-co-glycolic acid) microspheres: influence of manufacturing parameters and type of polymer on the release characteristics, J. Microencapsul. (1999) 16 (1): 1-12, each of which is incorporated herein in its entirety.

In yet further embodiments for delivery of aqueous-soluble bioactive agents, the particles can be made into nanoparticles having an average diameter of about 20 nm to about 200 nm for delivery to the circulation. The nanoparticles can be made by the single emulsion method with the active agent dispersed therein, i.e., mixed into the emulsion or conjugated to polymer as described herein. The nanoparticles can also be provided as a micellar composition containing the polymers described herein, such as PEA and PEUR with the bioactive agents conjugated thereto. Alternatively or in addition to bioactive agents conjugated to the polymers, since the micelles are formed in water, water soluble bioactive agents can be loaded into the micelles at the same time without solvent.

More particularly, the biodegradable micelles are formed of a hydrophobic polymer chain conjugated to a water soluble polymer chain. Whereas, the outer portion of the micelle mainly consists of the water soluble ionized or polar section of the polymer, the hydrophobic section of the polymer mainly partitions to the interior of the micelles and holds the polymer molecules together.

The biodegradable hydrophobic section of the polymer used to make micelles is made of PEEA, PEEUR or PEEU polymers, as described herein. For strongly hydrophobic PEEA, PEEUR or PEEU polymers, components such as di-L-leucine ester of 1,4:3,6-dianhydro-D-sorbitol or rigid aromatic di-acid like α,ω-bis(4-carboxyphenoxy) (C₁-C₈) alkane may be included in the polymer repeat unit. By contrast, the water soluble section of the polymer comprises repeating alternating units of i) polyethylene glycol having a Mw of at least 200 and less than, and b) at least one ionizable or polar amino acid, wherein the repeating alternating units have substantially similar molecular weights and wherein the Mw of the polymer is in the range from about 10 kD to about 300 kD. The repeating alternating units may have substantially similar molecular weights in the range from about 300 Da to about 700 Da. In one embodiment wherein the molecular weight of the polymer is over 10 kDa, at least one of the amino acid units is an ionizable or polar amino acid selected from serine, glutamic acid, aspartic acid, lysine and arginine. In one embodiment, the units of ionizable amino acids comprise at least one block of ionizable poly(amino acids), such as glutamate or aspartate, can be included in the polymer. The invention micellar composition may further comprise a pharmaceutically acceptable aqueous media with a pH value at which at least a portion of the ionizable amino acids in the water soluble sections of the polymer are ionized.

The higher the molecular weight of the water soluble segment of the polymer, the greater the porosity of the micelle and the higher the loading into the micelles of water soluble bioactive agents and/or large bioactive agents, such as proteins. In one embodiment, therefore, the molecular weight of the complete water soluble section of the polymer is in the range from about 5 kDa to about 100 kDa.

Once formed, the micelles can be lyophilized for storage and shipping and reconstituted in the above-described aqueous media. However, it is not recommended to lyophilize micelles containing certain bioactive agents, such as certain proteins, that would be denatured by the lyophilization process.

Charged moieties within the micelles partially separate from each other in water, and create space for absorption of water soluble agents, such as the bioactive agent(s). Ionized chains with the same type of charge will repel each other and create more space. The ionized polymer also attracts the bioactive agent, providing stability to the matrix. In addition, the water soluble exterior of the micelle prevents adhesion of the micelles to proteins in body fluids after ionized sites are taken by the therapeutic bioactive agent. This type of micelle has very high porosity, up to 95% of the micelle volume, allowing for high loading of aqueous-soluble biologics, such as polypeptides, DNA, and other bioactive agents. Particle size range of the micelles is about 20 nm to about 200 nm, with about 20 nm to about 100 nm being preferred for circulation in the blood.

Particle size can be determined by, e.g., dynamic light scattering, using for example, a spectrometer incorporating a helium-neon laser. Generally, particle size is determined at room temperature and involves multiple analyses of the sample in question (e.g., 5-10 times) to yield an average value for the particle diameter. Particle size is also readily determined using scanning electron microscopy (SEM). In order to do so, dry particles are sputter-coated with a gold/palladium mixture to a thickness of approximately 100 Angstroms, and then examined using a scanning electron microscope. Alternatively, the polymer, either in the form of particles or not, can be covalently attached directly to the bioactive agent, rather than incorporating bioactive agent therein (“loading) without chemical attachment, using any of several methods well known in the art and as described hereinbelow. The bioactive agent content is generally in an amount that represents approximately 0.1% to about 40% (w/w) bioactive agent to polymer, more preferably about 1% to about 25% (w/w) bioactive agent, and even more preferably about 2% to about 20% (w/w) bioactive agent. The percentage of bioactive agent will depend on the desired dose and the condition being treated, as discussed in more detail below.

Bioactive agents for dispersion into and release from the invention biodegradable polymer compositions also include anti-proliferants, rapamycin and any of its analogs or derivatives, paclitaxel or any of its taxene analogs or derivatives, everolimus, Sirolimus, Tacrolimus, or any of its—limus named family of drugs, and statins such as simvastatin, atorvastatin, fluvastatin, pravastatin, lovastatin, rosuvastatin, geldanamycins, such as 17AAG (17-allylamino-17-demethoxygeldanamycin); Epothilone D and other epothilones, 17-dimethylaminoethylamino-17-demethoxy-geldanamycin and other polyketide inhibitors of heat shock protein 90 (Hsp90), Cilostazol, and the like.

Additional bioactive agents contemplated for dispersion in the polymers used in the invention OEG-based polymer compositions include agents that, when freed or eluted from the polymer particles during their degradation, promote endogenous production of a therapeutic natural wound healing agent, such as nitric oxide, which is endogenously produced by endothelial cells. Alternatively the bioactive agents released from the polymers during degradation may be directly active in promoting natural wound healing processes by endothelial cells. These bioactive agents can be any agent that donates, transfers, or releases nitric oxide, elevates endogenous levels of nitric oxide, stimulates endogenous synthesis of nitric oxide, or serves as a substrate for nitric oxide synthase or that inhibits proliferation of smooth muscle cells. Such agents include, for example, aminoxyls, furoxans, nitrosothiols, nitrates and anthocyanins; nucleosides such as adenosine and nucleotides such as adenosine diphosphate (ADP) and adenosine triphosphate (ATP); neurotransmitter/neuromodulators such as acetylcholine and 5-hydroxytryptamine (serotonin/5-HT); histamine and catecholamines such as adrenalin and noradrenalin; lipid molecules such as sphingosine-1-phosphate and lysophosphatidic acid; amino acids such as arginine and lysine; peptides such as the bradykinins, substance P and calcium gene-related peptide (CGRP), and proteins such as insulin, vascular endothelial growth factor (VEGF), and thrombin.

A variety of bioactive agents, coating molecules and ligands for bioactive agents can be attached, for example covalently, to the surface of the polymer particles. Bioactive agents, such as targeting antibodies, polypeptides (e.g., antigens) and drugs, and the like, can be covalently conjugated to the surface of the polymer particles. In addition, coating molecules, such as polyethylene glycol (PEG) as a ligand for attachment of antibodies or polypeptides or phosphatidylcholine (PC) as a means of blocking attachment sites on the surface of the particles to prevent the particles from sticking to non-target biological molecules and surfaces in the patient may also be surface-conjugated.

For example, small proteinaceous motifs, such as the B domain of bacterial Protein A and the functionally equivalent region of Protein G are known to bind to, and thereby capture, antibody molecules by the Fc region. Such proteinaceous motifs can be attached to the polymers, especially to the surface of the polymer particles. Such molecules will act, for example, as ligands to attach antibodies for use as targeting ligands or to capture antibodies to hold precursor cells or capture cells out of the patient's blood stream. Therefore, the antibody types that can be attached to polymer coatings using a Protein A or Protein G functional region are those that contain an Fc region. The capture antibodies will in turn bind to and hold precursor cells, such as progenitor cells, near the polymer surface while the precursor cells, which are preferably bathed in a growth medium within the polymer, secrete various factors and interact with other cells of the subject. In addition, one or more bioactive agents dispersed in the polymer particles, such as the bradykinins, may activate the precursor cells.

In addition, bioactive agents for attaching precursor cells or for capturing progenitor endothelial cells (PECs) from the subject's blood are monoclonal antibodies directed against a known precursor cell surface marker. For example, complementary determinants (CDs) that have been reported to decorate the surface of endothelial cells include CD31, CD34, CD102, CD105, CD106, CD109, CDw130, CD141, CD142, CD143, CD144, CDw145, CD146, CD147, and CD166. These cell surface markers can be of varying specificity and the degree of specificity for a particular cell/developmental type/stage is in many cases not fully characterized. In addition these cell marker molecules against which antibodies have been raised will overlap (in terms of antibody recognition) especially with CDs on cells of the same lineage: monocytes in the case of endothelial cells. Circulating endothelial progenitor cells are some way along the developmental pathway from (bone marrow) monocytes to mature endothelial cells. CDs 106, 142 and 144 have been reported to mark mature endothelial cells with some specificity. CD34 is presently known to be specific for progenitor endothelial cells and therefore is currently preferred for capturing progenitor endothelial cells out of blood in the site into which the polymer particles are implanted for local delivery of the active agents. Examples of such antibodies include single-chain antibodies, chimeric antibodies, monoclonal antibodies, polyclonal antibodies, antibody fragments, Fab fragments, IgA, IgG, IgM, IgD, IgE and humanized antibodies.

The following additional bioactive agents and small molecule drugs will be particularly effective for dispersion within the invention polymer particle compositions, whether sized to form a time release biodegradable polymer depot for local delivery of the bioactive agents, or sized for entry into systemic circulation, as described herein. The bioactive agents that are dispersed in the polymer particles used in the invention compositions and methods of treatment will be selected for their suitable therapeutic or palliative effect in treatment of a disease of interest, or symptoms thereof.

In one embodiment, the suitable bioactive agents are not limited to, but include, various classes of compounds that facilitate or contribute to wound healing when presented in a time-release fashion. Such bioactive agents include wound-healing cells, including certain precursor cells, which can be protected and delivered by the biodegradable polymer particles in the invention compositions. Such wound healing cells include, for example, pericytes and endothelial cells, as well as inflammatory healing cells. To recruit such cells to the site of a polymer depot in vivo, the polymer particles used in the invention compositions and methods of treatment can include ligands for such cells, such as antibodies and smaller molecule ligands, that specifically bind to “cellular adhesion molecules” (CAMs). Exemplary ligands for wound healing cells include those that specifically bind to Intercellular adhesion molecules (ICAMs), such as ICAM-1 (CD54 antigen); ICAM-2 (CD102 antigen); ICAM-3 (CD50 antigen); ICAM-4 (CD242 antigen); and ICAM-5; Vascular cell adhesion molecules (VCAMs), such as VCAM-1 (CD106 antigen)]; Neural cell adhesion molecules (NCAMs), such as NCAM-1 (CD56 antigen); or NCAM-2; Platelet endothelial cell adhesion molecules PECAMs, such as PECAM-1 (CD31 antigen); Leukocyte-endothelial cell adhesion molecules (ELAMs), such as LECAM-1; or LECAM-2 (CD62E antigen), and the like.

In another aspect, the suitable bioactive agents include extra cellular matrix proteins, macromolecules that can be dispersed into the polymer particles used in the invention compositions, e.g., attached either covalently or non-covalently. Examples of useful extra-cellular matrix proteins include, for example, glycosaminoglycans, usually linked to proteins (proteoglycans), and fibrous proteins (e.g., collagen; elastin; fibronectins and laminin). Bio-mimics of extra-cellular proteins can also be used. These are usually non-human, but biocompatible, glycoproteins, such as alginates and chitin derivatives. Wound healing peptides that are specific fragments of such extra-cellular matrix proteins and/or their bio-mimics can also be used as the bioactive agent.

Proteinaceous growth factors are an additional category of bioactive agents suitable for dispersion in the polymer particles used in the invention compositions and methods of treatment described herein. Such bioactive agents are effective in promoting wound healing and other disease states as is known in the art. For example, Platelet Derived Growth Factor-BB (PDGF-BB), Tumor Necrosis Factor-alpha (TNF-α), Epidermal Growth Factor (EGF), Keratinocyte Growth Factor (KGF), Thymosin B4; and, various angiogenic factors such as vascular Endothelial Growth Factors (VEGFs), Fibroblast Growth Factors (FGFs), Tumor Necrosis Factor-beta (TNF-beta), and Insulin-like Growth Factor-1 (IGF-1). Many of these proteinaceous growth factors are available commercially or can be produced recombinantly using techniques well known in the art.

Alternatively, expression systems comprising vectors, particularly adenovirus vectors, incorporating genes encoding a variety of biomolecules can be dispersed in the polymer particles for timed release delivery. Method of preparing such expression systems and vector are well known in the art. For example, proteinaceous growth factors can be dispersed into the invention polymer particles for administration of the growth factors either to a desired body site for local delivery by selection of particles sized to form a polymer depot or systemically by selection of particles of a size that will enter the circulation. The growth factors such as VEGFs, PDGFs, FGF, NGF, and evolutionary and functionally related biologics, and angiogenic enzymes, such as thrombin, may also be used as bioactive agents in the invention.

Organic or inorganic chemical compounds “small molecule drugs” are an additional category of bioactive agents suitable for dispersion in the polymer particles used in the invention compositions and methods of treatment described herein. Such drugs include, for example, antimicrobials and anti-inflammatory agents as well as certain healing promoters, such as, for example, vitamin A and synthetic inhibitors of lipid peroxidation.

A variety of antibiotics can be dispersed in the polymer particles used in the invention compositions to indirectly promote natural healing processes by preventing or controlling infection. Suitable antibiotics include many classes, such as aminoglycoside antibiotics or quinolones or beta-lactams, such as cefalosporins, ciprofloxacin, gentamycin, tobramycin, erythromycin, vancomycin, oxacillin, cloxacillin, methicillin, lincomycin, ampicillin, and colistin. Suitable antibiotics have been described in the literature.

Suitable antimicrobials include, for example, Adriamycin PFS/RDF® (Pharmacia and Upjohn), Blenoxane® (Bristol-Myers Squibb Oncology/Immunology), Cerubidine® (Bedford), Cosmegen® (Merck), DaunoXome® (NeXstar), Doxil® (Sequus), Doxorubicin Hydrochloride® (Astra), Idamycin® PFS (Pharmacia and Upjohn), Mithracin® (Bayer), Mitamycin® (Bristol-Myers Squibb Oncology/Immunology), Nipen® (SuperGen), Novantrone® (Immunex) and Rubex® (Bristol-Myers Squibb Oncology/Immunology). In one embodiment, the peptide can be a glycopeptide. “Glycopeptide” refers to oligopeptide (e.g. heptapeptide) antibiotics, characterized by a multi-ring peptide core optionally substituted with saccharide groups, such as vancomycin.

Examples of glycopeptides included in this category of antimicrobials may be found in “Glycopeptides Classification, Occurrence, and Discovery,” by Raymond C. Rao and Louise W. Crandall, in Bioactive agents and the Pharmaceutical Sciences” Volume 63, edited by Ramakrishnan Nagarajan, published by Marcal Dekker, Inc.). Additional examples of glycopeptides are disclosed in U.S. Pat. Nos. 4,639,433; 4,643,987; 4,497,802; 4,698,327, 5,591,714; 5,840,684; and 5,843,889; in EP 0 802 199; EP 0 801 075; EP 0 667 353; WO 97/28812; WO 97/38702; WO 98/52589; WO 98/52592; and in J. Amer. Chem. Soc. (1996) 118:13107-13108; J. Amer. Chem. Soc. (1997) 119: 12041-12047; and J. Amer. Chem. Soc. (1994) 116:4573-4590. Representative glycopeptides include those identified as A477, A35512, A40926, A41030, A42867, A47934, A80407, A82846, A83850, A84575, AB-65, Actaplanin, Actinoidin, Ardacin, Avoparcin, Azureomycin, Balhimyein, Chloroorientiein, Chloropolysporin, Decaplanin, N-demethylvancomycin, Eremomycin, Galacardin, Helvecardin, Izupeptin, Kibdelin, LL-AM374, Mannopeptin, MM45289, MM47756, MM47761, MM49721, MM47766, MM55260, MM55266, MM55270, MM56597, MM56598, OA-7653, Orenticin, Parvodicin, Ristocetin, Ristomycin, Synmonicin, Teicoplanin, UK-68597, UD-69542, UK-72051, Vancomycin, and the like. The term “glycopeptide” or “glycopeptide antibiotic” as used herein is also intended to include the general class of glycopeptides disclosed above on which the sugar moiety is absent, i.e. the aglycone series of glycopeptides. For example, removal of the disaccharide moiety appended to the phenol on vancomycin by mild hydrolysis gives vancomycin aglycone. Also included within the scope of the term “glycopeptide antibiotics” are synthetic derivatives of the general class of glycopeptides disclosed above, included alkylated and acylated derivatives. Additionally, within the scope of this term are glycopeptides that have been further appended with additional saccharide residues, especially aminoglycosides, in a manner similar to vancosamine.

The term “lipidated glycopeptide” refers specifically to those glycopeptide antibiotics that have been synthetically modified to contain a lipid substituent. As used herein, the term “lipid substituent” refers to any substituent contains 5 or more carbon atoms, preferably, 10 to 40 carbon atoms. The lipid substituent may optionally contain from 1 to 6 heteroatoms selected from halo, oxygen, nitrogen, sulfur, and phosphorous. Lipidated glycopeptide antibiotics are well known in the art. See, for example, in U.S. Pat. Nos. 5,840,684, 5,843,889, 5,916,873, 5,919,756, 5,952,310, 5,977,062, 5,977,063, EP 667, 353, WO 98/52589, WO 99/56760, WO 00/04044, WO 00/39156, the disclosures of which are incorporated herein by reference in their entirety.

Anti-inflammatory bioactive agents are also useful for dispersion in polymer particles used in invention compositions and methods. Depending on the body site and disease to be treated, such anti-inflammatory bioactive agents include, e.g. analgesics (e.g., NSAIDS and salicyclates), steroids, antirheumatic agents, gastrointestinal agents, gout preparations, hormones (glucocorticoids), nasal preparations, ophthalmic preparations, otic preparations (e.g., antibiotic and steroid combinations), respiratory agents, and skin and mucous membrane agents. See, Physician's Desk Reference, 2001 Edition. Specifically, the anti-inflammatory agent can include dexamethasone, which is chemically designated as (11θ, 16I)-9-fluro-11,17,21-trihydroxy-16-methylpregna-1,4-diene-3,20-dione. Alternatively, the anti-inflammatory bioactive agent can be or include sirolimus (rapamycin), which is a triene macrolide antibiotic isolated from Streptomyces hygroscopicus.

The polypeptide bioactive agents included in the invention compositions and methods can also include “peptide mimetics.” Such peptide analogs, referred to herein as “peptide mimetics” or “peptidomimetics,” are commonly used in the pharmaceutical industry with properties analogous to those of the template peptide (Fauchere, J. (1986) Adv. Bioactive agent Res., 15:29; Veber and Freidinger (1985) TINS, p. 392; and Evans et al. (1987) J. Med. Chem., 30:1229) and are usually developed with the aid of computerized molecular modeling. Generally, peptidomimetics are structurally similar to a paradigm polypeptide (i.e., a polypeptide that has a biochemical property or pharmacological activity), but have one or more peptide linkages optionally replaced by a linkage selected from the group consisting of: —CH₂NH—, —CH₂S—, CH₂—CH₂—, —CH═CH— (cis and trans), —COCH₂—, —CH(OH)CH₂—, and —CH₂SO—, by methods known in the art and further described in the following references: Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), 1(3), “Peptide Backbone Modifications” (general review); Morley, J. S., Trends. Pharm. Sci., (1980) pp. 463-468 (general review); Hudson, D. et al., Int. J. Pept. Prot. Res., (1979) 14:177-185 (—CH₂NH—, CH₂CH₂—); Spatola, A. F. et al., Life Sci., (1986) 38:1243-1249 (—CH₂—S—); Harm, M. M., J. Chem. Soc. Perkin Trans 1(1982) 307-314 (—CH═CH—, cis and trans); Almquist, R. G. et al., J. Med. Chem., (1980) 23:2533 (—COCH₂—); Jennings-Whie, C. et al., Tetrahedron Lett., (1982) 23:2533 (—COCH₂—); Szelke, M. et al., European Appin., EP 45665 (1982) CA: 97:39405 (1982) (—CH(OH)CH₂—); Holladay, M. W. et al., Tetrahedron Lett., (1983) 24:4401-4404 (—C(OH)CH₂—); and Hruby, V. J., Life Sci., (1982) 31:189-199 (—CH₂—S—). Such peptide mimetics may have significant advantages over natural polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others.

Additionally, substitution of one or more amino acids within a peptide (e.g., with a D-Lysine in place of L-Lysine) may be used to generate more stable peptides and peptides resistant to endogenous peptidases. Alternatively, the synthetic polypeptides covalently bound to the biodegradable polymer, can also be prepared from D-amino acids, referred to as inverso peptides. When a peptide is assembled in the opposite direction of the native peptide sequence, it is referred to as a retro peptide. In general, polypeptides prepared from D-amino acids are very stable to enzymatic hydrolysis. Many cases have been reported of preserved biological activities for retro-inverso or partial retro-inverso polypeptides (U.S. Pat. No. 6,261,569 B1 and references therein; B. Fromme et al, Endocrinology (2003)144:3262-3269.

It is readily apparent that the subject invention can be used to prepare compositions to be used in preventing or treating a wide variety of diseases or symptoms thereof.

Following preparation of the polymer particles loaded with bioactive agent, the composition can be lyophilized and the dried composition suspended in an appropriate media prior to administration.

Any suitable and effective amount of the at least one active agent can be released with time from the polymer particles (including those in a polymer depot formed in vivo) and will typically depend, e.g., on the specific polymer, type of particle or polymer/bioactive agent linkage, if present. Typically, up to about 100% of the polymer particles can be released from a polymer depot formed in vivo by particles sized to avoid circulation. Specifically, up to about 90%, up to 75%, up to 50%, or up to 25% thereof can be released from the polymer depot. Factors that typically affect the release rate from the polymer are the nature and amount of the polymer/bioactive agent, the types of polymer/bioactive agent linkage, and the nature and amount of additional substances present in the formulation.

Once the invention OEG-based polymer composition is made, as above, compositions are formulated for subsequent intrapulmonary, gastroenteral, subcutaneous, intramuscular, into the central nervous system, intraperitoneum or intraorgan delivery. The compositions will generally include one or more “pharmaceutically acceptable excipients or vehicles” appropriate for oral, mucosal or subcutaneous delivery, such as water, saline, glycerol, polyethylene glycol, hyaluronic acid, ethanol, and the like. Additionally, auxiliary substances, such as wetting or emulsifying agents, pH buffering substances, flavorings, and the like, may be present in such vehicles. For example, excipients, such as detergents, may serve various purposes: as an emulsion stabilizer, modifier of interfacial tension or to increase the load of less soluble drug into particles. Examples of excipients suitable for use in the invention compositions include polyvinyl alcohol (PVA), TWEEN® 80, Pluronic F-68, sodium dodecyl sulfate (SDS), Brij®-35, and N-dodecyl-β-D-maltoside, octyl-β-D-glucopyranoside, IGEPAL® CA-630, N-octanoyl-N-methylglucamine, N-nonanoyl-N-methylglucamine, N-decanoyl-N-methylglucamine, Nonidet® P-40 substitute, Saponin, hexadecylmethyl ammonium bromide, CHAPS, EMPIGEN® BB, and 3-(Dodecyldimethylammonio) propane sulfonate inner salt (SB3-12), all of which are commercially available.

Intranasal and pulmonary formulations will usually include vehicles that neither cause irritation to the nasal mucosa nor significantly disturb ciliary function. Diluents such as water, aqueous saline or other known substances can be employed with the subject invention. The intrapulmonary formulations may also contain preservatives such as, but not limited to, chlorobutanol and benzalkonium chloride. A surfactant may be present to enhance absorption by the nasal mucosa.

For rectal and urethral suppositories, the vehicle used in the invention compositions may include traditional binders and carriers, such as, cocoa butter (theobroma oil) or other triglycerides, vegetable oils modified by esterification, hydrogenation and/or fractionation, glycerinated gelatin, polyalkaline glycols, mixtures of polyethylene glycols of various molecular weights and fatty acid esters of polyethylene glycol.

For vaginal delivery, the compositions of the present invention can be incorporated in or may include pessary bases, such as those including mixtures of polyethylene triglycerides, or suspended in oils such as corn oil or sesame oil, optionally containing colloidal silica. See, e.g., Richardson et al., Int. J. Pharm. (1995) 115:9-15.

For a further discussion of appropriate vehicles to use for particular modes of delivery, see, e.g., Remington: The Science and Practice of Pharmacy, Mack Publishing Company, Easton, Pa., 19th edition, 1995. One of skill in the art can readily determine the proper vehicle to use for the particular bioactive agent/polymer particle combination, size of particle and mode of administration.

In addition to treatment of humans, the invention OEG-based polymer compositions are also intended for use in veterinary treatment of a variety of mammalian patients, such as pets (for example, cats, dogs, rabbits, and ferrets), farm animals (for example, swine, horses, mules, dairy and meat cattle) and race horses.

The compositions used in the invention methods optionally may comprise an “effective amount” of the bioactive agent(s) of interest dispersed in the invention OEG-based PE PEEA, PEEUR or PEEU polymer. That is, an amount of a bioactive agent may be included in the compositions that will cause the subject to produce a sufficient therapeutic or palliative response in order to prevent, reduce or eliminate symptoms. The exact amount necessary will vary, depending on the subject being treated; the age and general condition of the subject to be treated; the capacity of the subject's immune system, the degree of treatment desired; the severity of the condition being treated; the particular bioactive agent selected, and the mode of administration of the composition, among other factors. An appropriate effective amount can be readily determined by one of skill in the art. Thus, an “effective amount” will fall in a relatively broad range that can be determined through routine trials. For example, for purposes of the present invention, an effective amount will typically range from about 1 μg to about 100 mg, for example from about 5 μg to about 1 mg, or about 10 μg to about 500 μg of the active agent delivered per dose.

Once formulated, the invention OEG-based polymer compositions are administered orally, mucosally, or by subcutaneously or intramuscular injection, and the like, using standard techniques. See, e.g., Remington: The Science and Practice of Pharmacy, supra, for mucosal delivery techniques, including intranasal, pulmonary, vaginal and rectal techniques, as well as European Publication No. 517,565 and Ilium et al., J. Controlled Rel. (1994) 29:133-141, for techniques of intranasal administration.

Dosage treatment may be a single dose of the invention OEG-based polymer composition, or a multiple dose schedule as is known in the art. The dosage regimen, at least in part, will also be determined by the need of the subject and be dependent on the judgment of the practitioner. Furthermore, if prevention of disease is desired, the polymer composition is generally administered prior to primary disease manifestation, or symptoms of the disease of interest. If treatment is desired, e.g., the reduction of symptoms or recurrences, the polymer compositions are generally administered subsequent to primary disease manifestation.

The formulations can be tested in vivo in a number of animal models developed for the study of oral subcutaneous or mucosal delivery. For example, the conscious sheep model is an art-recognized model for testing nasal delivery of substances See, e.g., Longenecker et al., J. Pharm. Sci. (1987) 76:351-355 and Ilium et al., J. Controlled Rel. (1994) 29:133-141. The polymer composition, generally in powdered, lyophilized form, is blown into the nasal cavity. Blood samples can be assayed for active agent using standard techniques, as known in the art.

To illustrate the invention, a series of PEEAs were synthesized. First, three different types of di-p-nitrophenyl esters of dicarboxylic acids, NA, NS and NF, were synthesized as monomers to provide the carboxylic ester segment of PEEA. NA and NS have saturated methylene structure, while NF has unsaturated C═C double bonds in the methylene structure (See Scheme IV).

As the monomers to provide the amino acid segment of the PEEAs, three different types of di-p-toluenesulfonic acid salts of bis-L-phenylalanine diesters were synthesized as described in the Example herein. All three salts contained residues of oligomers of ethylene glycol with ether bonds in the segment (Scheme V)

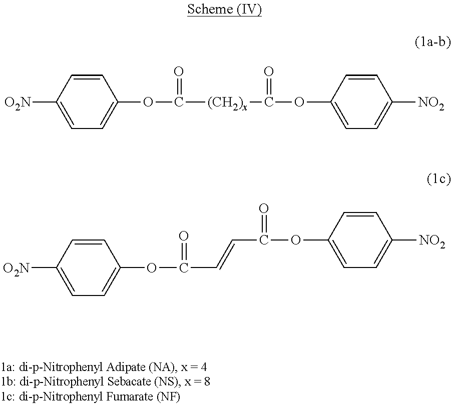

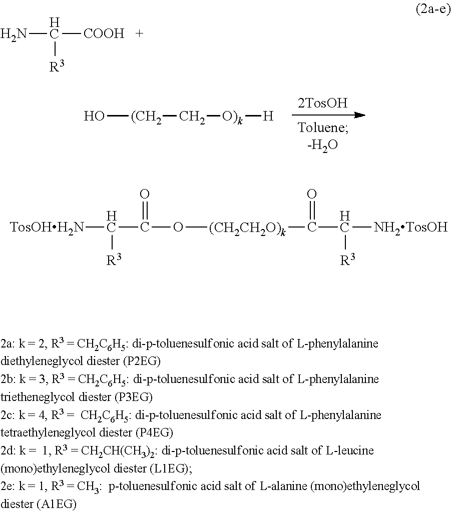

The chemical structures of the di-p-toluenesulfonic acid salt monomers 2 a-c, referred to herein as P2EG, P3EC and P4EG, were all confirmed by elemental analysis, FTIR and ¹H- and ¹³C-NMR spectra. The FTIR spectra showed the existence of both the primary amine salt, ester carbonyl —CO—, at 1741 cm⁻¹ and the characteristic signals of —CH₂—O—CH₂— in 1127 cm⁻¹ region.

Synthesis of the illustrative examples of the invention OEG-based PEEAs was by solution polycondensation of different combinations of monomer 1 (1a, 1b or 1c) and monomer 2 (2a, 2b or 2c), as shown in Scheme (VI) below.

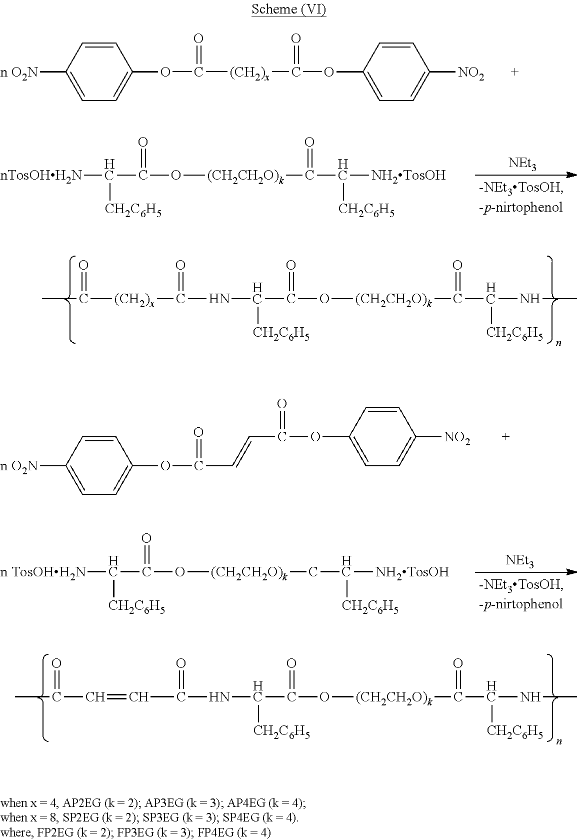

The chemical structures of these PEEA polymers were confirmed by examination of both FTIR and NMR spectra. The FTIR spectra of all the fumaryl-based PEEAs confirmed the existence of unsaturated —C═C— bonds (983 cm⁻¹), as well as ester carbonyls (1740 cm⁻¹), ether groups 1115 cm⁻¹), amide groups (1640 and 1530 cm⁻¹). The NMR spectra of three typical PEEAs based on tri-ethylene glycol were fully in agreement with the anticipated chemical structures of the PEEA polymers shown in Scheme VI.

Nine different types of PEEAs were synthesized by solution polycondensation of combinations of monomer 1a-c and 2 a-c and entered in Table 1.

TABLE 1
Properties of poly(ether ester amide)s^{a)} of Formula I

| | Yield | η_{red} | Mn | Mw | | | |
|---|---|---|---|---|---|---|---|
| Polymer | (%) | (dL/g)^{b} | (kg/mol) | (kg/mol) | Mw/Mn | Tg (° C.) | Tm (° C.) |
| FP2EG^{c} | 76 | 0.13 ± 0.01 | — | — | — | 82 | 233 |
| FP3EG^{c} | 83 | 0.49 ± 0.01 | — | — | — | 67 | 180 |
| FP4EG^{c} | 69 | 0.27 ± 0.00 | — | — | — | 54 | 138 |
| AP2EG | 59 | 0.13 ± 0.01 | 2.6 | 4.6 | 1.76 | 49 | 101 |
| AP3EG | 79 | 0.61 ± 0.03 | 17.4 | 26.0 | 1.49 | 35 | 92 |
| AP4EG | 74 | 0.24 ± 0.01 | 9.0 | 14.6 | 1.63 | 14 | 63 |
| SP2EG | 62 | 0.16 ± −/−1 | 6.4 | 10.2 | 1.59 | 32 | 90 |
| SP3EG | 74 | 0.54 ± 0.00 | 27.3 | 41.1 | 1.51 | 23 | 67 |
| SP4EG | 63 | 0.17 ± 0.00 | 7.3 | 118.5 | 2.53 | 12 | 58 |

^{a)}Synthesis conditions: c = 0.90 mol/L. t = 70° C., THF as the solvent
^{b}Measured in DMSO at 25° C., C = 0.25 g/dL.
^{c}Molecular weight data not available for fumaric acid based PEEA due to insolubility in THF A—adipic, F—fumaric and S—sebacis acids; P = L-Phe; K = 2, 3, 4 in scheme 6;

Among the adipic and sebacic acid based PEEAs, AP3EG and SP3EG achieved the highest Mₙ, M_{w} and reduced viscosity η_{red}.

The synthesized PEEAs had a lower T_{g} than the corresponding saturated or unsaturated PEAs containing the residue of aliphatic diol (Katsasava, R. et al. J Polym Sci Pol Chem (1999) 37(4):391-407 and Guo et al, supra). In fact, within each PEEA series, an increase in the number of ether bonds led to a corresponding reduction in T_{g.} and it appeared more pronounced in the saturated PEEA series than in unsaturated PEEAs. Thus, the lower T_{g} of the PEEAs was attributed to the presence of ether bonds, causing increased chain flexibility and promoting chain segmental movement.

The biodegradation kinetics of two representative invention triethyleneglycol-based PEEA polymers (AP3EG and SP3EG) were studied in pH 7.4 PBS buffer and α-chymotrypsin buffer solution at 37° C. Both polymers have exhibited much faster weight loss in α-chymotrypsin solution than in a PBS buffer, and the level of biodegradation of these PEEA polymers in an enzyme solution depended on both the chemical structures and the enzyme concentration. Of the polymers tested, AP3EG was most sensitive to enzymatic biodegradation (FIG. 2).

Moreover, irrespective of different α-chymotrypsin concentrations used in the biodegradation studies, the weight loss kinetics of the exemplary OEG-based PEEAs were very close to zero-order attributed to the surface erosion.

To illustrate synthesis of the invention PEEURs, the following monomers—active bis-p-nitrophenyl carbonates containing ether blocks of various lengths—were synthesized as described in Example 2 herein. Individual ether diols, di-, tri-, tetra-ethyleneglycols, are commercially available and suitable for this task. Active bis-p-nitrophenyl carbonates were synthesized according to general scheme (VII)

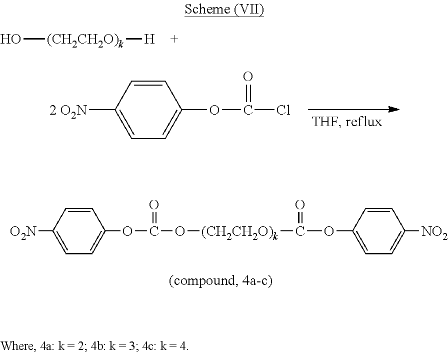

Chemical structures of obtained monomers were confirmed by elemental analysis, ¹H NMR and identified with reported melting points. Synthesis of illustrative examples of the invention OEG-based PEEURs was conducted by solution polycondensation method analogous to above described PEEA synthesis, as shown in scheme (VIII) below.

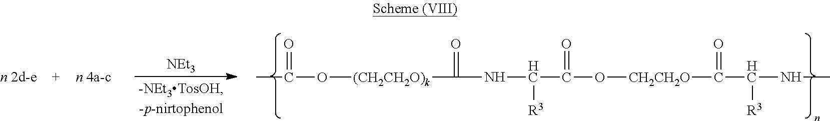

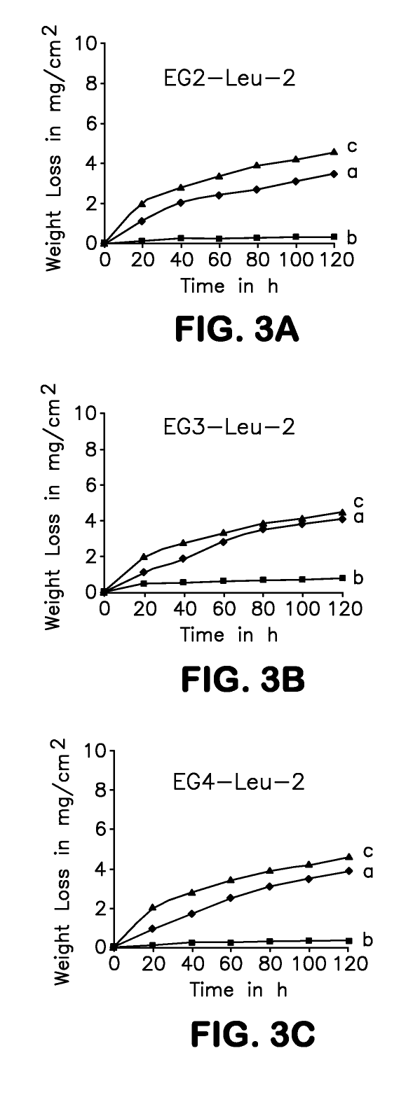

Weight average molecular weights of the synthesized PEEURs ranged within 31,400-51,000 g/mol, as estimated by GPC, (DMF, PMMA), with glass transition temperature in the range of 12-39° C. Lipase catalyzed biodegradation was conducted on L1EG based PEEURs. Results are illustrated in FIGS. 3A-C, in which the data show that obtained PEEURs were not subjected to chemical hydrolysis in phosphate buffer. By contrast. rates of lipase catalyzed biodegradation were high and not dependent on the length of OEG fragment in R⁶ of formulas V-VI.

The following Examples are meant to illustrate, but not to limit the invention.

### Example 1

In this example, preparation and testing of a series of saturated and unsaturated OEG-based PEAs (PEEAs) is described to illustrate the properties of the invention compositions.

### A. Preparation of Materials

L-Phenylalanine (L-Phe), p-toluenesulfonic acid monohydrate (TosOH.H₂O), sebacoyl chloride, adipoyl chloride, fumaryl chloride, di-ethylene glycol, tri-ethylene glycol and tetra-ethylene glycol (Alfa Aesar, Ward Hill, Mass.), and p-nitrophenol (J. T. Baker, Phillipsburg, N.J.) were used without further purification. Triethylamine (Fisher Scientific, Fairlawn, N.J.) was dried by refluxing with calcium hydride, and then distilled. N,N-Dimethylformamide (DMF) (Aldrich Chemical, Milwaukee, Wis.) was dried over calcium hydride and distilled. Other solvents, such as toluene, trifluoroethanol (TFE), tetrahydrofuran (THF), ethyl acetate, acetone, acetonitrile, N,N-dimethylacetamide (DMA), and dimethyl sulfoxide (DMSO) were purchased from VWR Scientific (West Chester, Pa.) and were purified by standard methods before use.

### B. Synthesis of Monomers and Polymers

Following monomers were prepared for PEEA synthesis of Formula (I) and (IV): Di-p-nitrophenyl esters of dicarboxylic acids (1a-c), and di-p-toluenesulfonic acid salts of bis-L-phenylalanine-glycol-esters (2a-c);

Synthesis of di-p-nitrophenyl esters of dicarboxylic acids (compound 1a-c) were conducted by reacting corresponding acyl chlorides with p-nitrophenol as described previously (U.S. Pat. No. 6,503,538 B1, and Guo, K., et al. J. Polym Sci Poi Chem (2005) 43(7):1463-1477). Briefly, a solution of triethylamine (0.0603 mol) and p-nitrophenol (0.0603 mol) in 100 mL acetone was prepared at room temperature and this solution was kept at −78° C. by dry ice and acetone. Fumaryl chloride (0.03 mol, 3.2 mL) in 40 mL acetone was then added dropwise into the above chilled solution with stirring for 2 hr at −78° C. and then stirring was continued at room temperature overnight. After that, the mixture was poured into 800 mL of distilled water to precipitate the product, di-p-nitrophenyl fumarate (1c), which was filtered, washed thoroughly with distilled water, dried in vacuo at 50° C., and finally purified by recrystallization from acetonitrile.

Di-p-Toluenesulfonic Acid Salts of bis-L-Phenylalanine Esters (compounds 2a-c), were prepared by modifying procedures described in Katsarava at all, 1999, supra) as shown in scheme (V) previously.

Typically, L-Phe (0.176 mol), p-toluenesulfonic acid monohydrate (0.176 mol) and glycol (0.08 mol) in 300 mL of toluene were placed in a flask equipped with a Dean-Stark apparatus, a CaC1₂ drying tube, and a magnetic stirrer. The reaction mixture was heated (c.a. 140° C.) to reflux, until 6.1 mL (0.34 mol) of water evolved, then cooled to room temperature, solvent was removed by rotary evaporation, the mixture was dried in vacuo overnight and finally purified by recrystallization. Compound 2a (P2EG) was re-crystallized in water, and compounds 2b and 2c were crystallized from 2-propanol.

Solution Polycondensation of monomers 1 and 2: PEEAs were prepared by solution polycondensation of 2a-c with one of the di-p-nitrophenyl esters (1a-c), according to scheme (VI). The polymer composition and designations are shown in scheme VI and in Table 3 (PEEAs starting with F, such as FP3EG, were unsaturated based on fumaryl moieties;).

An example of OEG-containing polymer AP2EG synthesis via solution polycondensation is presented: Ten millimoles (1.42 mL) triethylamine was added dropwise to the mixture of monomers 1a, 4.0 mmol and 2a, 4.0 mmol in 3 mL of dry DMA, and the solution was heated to 60° C. with stirring until complete dissolution of monomers. The reaction vial was then kept at less than 70° C. for 48 hrs without stirring. The resulting viscous solution (from light yellow to dark brown in color depending on the type of monomers used) was divided into two groups and precipitated by different solvents. Unsaturated PEEA reaction mixtures (FP2EG9, FP3EG and FP4EG), were diluted with acetone to precipitate the product. The polymer was then washed by acetone twice, filtered and fmally dried in vacuo for 48 hr.

Saturated PEEAs were precipitated out in chilled ethyl acetate, products were filtered and further purified in a Soxhlet apparatus by circulating ethyl acetate for 48 h, and dried in vacuo for 48 hr.

Polymer solubility. Almost all PEEAs were soluble in DMSO, DMF and formic acid, except FP2EC, but would not dissolve in water, methanol and ethyl acetate. Saturated PEEAs also dissolve in trifluoroethanol, THF and chloroform. However, the three unsaturated PEEAs were only slightly soluble in those regular organic solvents. Thus, compared with PEA polymers derived from aliphatic diols, the OEG-based PEEAs, especially unsaturated PEEAs, showed a significant improvement in solubility. For example, SP3EC and SP4EG, both dissolved in acetone, while SPB (1,4-butanediol-based PEA) and SPH (1,6-hexanediol-based PEA) were insoluble.

### C. Testing Methods Used to Confirm the Chemical Structures and Properties of the Monomers and Polymers.

Chemical structures were confirmed by Fourier transform infrared (FTIR) and NMR spectra. For FTIR characterization, samples were ground into powder and mixed with KBr at a ratio of 1:10 w/w. FTIR spectra of the samples were then obtained using a Perkin-Elmer Nicolet Magana 560 (Madison, Wis.) FTIR spectrometer with Omnic software for data acquisition and analysis. NMR spectra of the samples were recorded by a Varian Unity NOVA-400 400 MHz spectrometer (Palo Alto, Calif.) operating at 400 and 100 MHz for ¹H and ¹³C NMR, respectively. Deuterated dimethyl sulfoxide (DMSO-d₆, Cambridge Isotope laboratories) was used as solvent. Elemental analyses of the polymers synthesized were performed using a PE 2400 CHN elemental analyzer (Atlantic Microlab, Norcross, Ga.).

Thermal properties of synthesized monomers and polymers were characterized by differential scanning calorimeter DSC 2920 (TA Instruments, New Castle, Del.). The measurement was carried out from 0° C. to 300° C. at a scanning rate of 10° C./min and nitrogen gas flow rate of 25 mL/min. TA Universal Analysis™ software was used for thermal data analysis, such as the determination of glass transition temperature. The melting point was determined at the onset of the melting endotherm.

The number and weight average molecular weights and molecular weight distributions (MWD) of synthesized PEEAs were determined by gel permeation chromatography (Model 510, Waters Associates Inc. Milford, USA) equipped with a high-pressure liquid chromatographic pump, a Waters 486 UV detector and a Waters 2410 differential refractive index detector. Tetrahydrofuran (THF) was used as eluent (1.0 mL/min). The columns were calibrated with polystyrene standards having a narrow molecular weight distribution. The reduced viscosity (η_{red}) of the polymers synthesized was determined by a Cannon-Ubbelhode viscometer in DMSO solution at a concentration of 0.25 g/dL at 25° C.

For biodegradability study the PEEA films were cast from 10% (wt/v) chloroform solution onto Teflon Petri dishes and allowed solvent to evaporate completely at room temperature. The films were further dried in vacuo at room temperature overnight and finally punched into small disc with diameter 12.5 mm.

In vitro biodegradation testing of OEG-based PEEAs was carried out in a small vial containing a disc of dry PEEA film, and 10 mL of pure PBS buffer or α-chymotrypsin solution in PBS buffer (pH=7.4, 0.1 M) of different concentrations (0, 0.05, 0.1 or 0.2 mg/mL). Two representative OEG-based PEEA polymers (AP3EG and SP3EG) were used for this biodegradation study. Samples were then incubated at 37° C. with constant reciprocal shaking (100 rpm). At predetermined intervals, PEEA films were removed from the incubation medium, washed gently with distilled water, surface water was blotted by filter paper, and weighed. The degree of biodegradation was estimated from the weight-loss of the PEEA film sample based on the following equation:

$${W}_{l} ( \% ) = \frac{{W}_{o} - {W}_{t}}{{W}_{o}} \times 100$$

wherein Wₒ, is the original weight of the dry PEEA film sample before immersion, and Wₜ is the dry PEEA film sample weight after incubation for t hrs (with or without enzyme). The weight loss average of three specimens was recorded.

The molecular weight changes of the PEEA polymers were also monitored by GPC.

The surface hydrophilicity of the PEEA films was determined by using a MASS Contact Angle Analyzer in a conditioning room maintained at 65% relative humidity and 21° C. Distilled water was used as the spreading liquid and the contact angle at five randomly chosen surface areas of each PEEA film was measured. Two PEEA films of each sample type were tested.

Confirmation of structures of Monomer 2 The chemical structures of the di-p-toluenesulfonic acid salts monomers, 2a-e, were all confirmed by analysis of FTIR and NMR spectra. In the FTIR spectra, main absorption bands, such as ester groups (1736-1750 cm⁻¹) and ether groups (about 1127 cm⁻¹) were assigned. A broad absorption between 2500 and 3300 cm⁻¹ could be attributed to both the primary amine salt and to aliphatic hydrocarbon structure existing in the ester salts. The ¹H NMR data of 2a-e also showed the characteristic signals of —CH₂—O—CH₂—.

Confirmation of structures for polymers. The FTIR spectra of fumaryl-based PEEAs exhibited the characteristic absorption bands of ester groups (˜1740 cm⁻¹), ether groups (˜1115 cm⁻¹), amide groups (˜1640 cm⁻¹ and ˜1530 cm⁻¹) and unsaturated —C∇C— bonds (˜983 cm⁻¹). The broad absorption around 2900 cm⁻¹ could be attributed to aliphatic hydrocarbon structure existing in the polymers. The ¹H-NMR spectra (400 MHz, DMSO-d₆) of the PEEAs showed proton signals of the —NH— bonds (8.90 or 8.24 ppm), the ether CH₂—O—CH₂ bonds in the diester unit (about 3.50 ppm) and —HC═CH— bonds in the acid unit (6.83 ppm). The data from elemental analysis were also consistent with those calculated for the compositions.

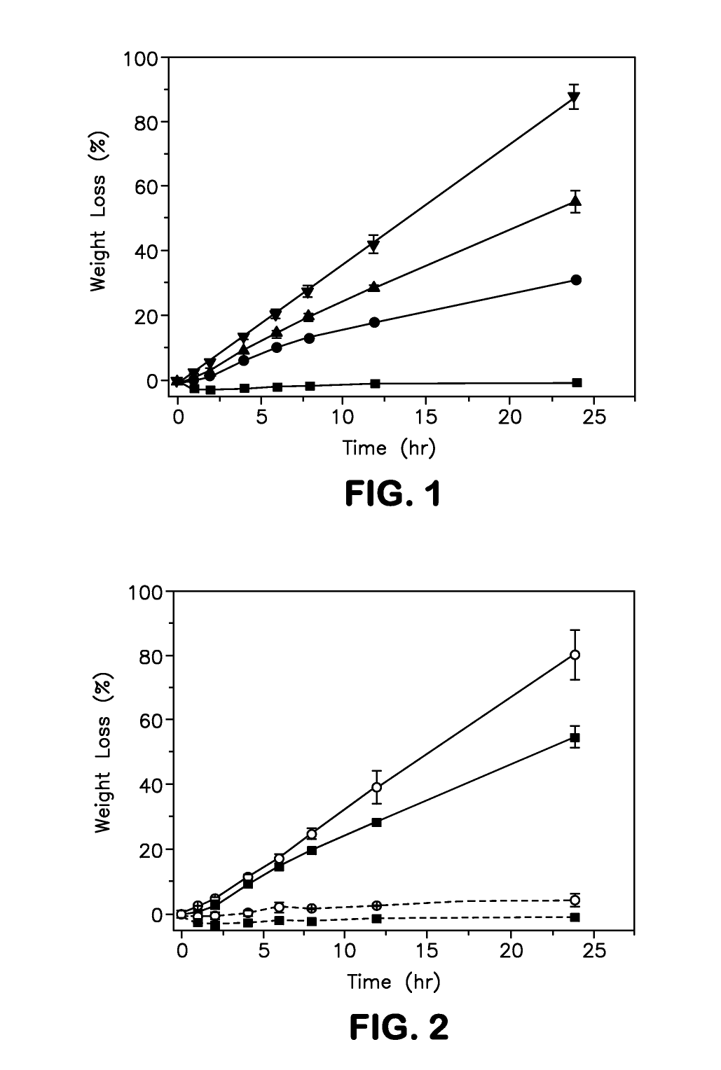

Results of biodegradation studies The biodegradation kinetics of two representatives OEG-based PEEA polymers (AP3EG and SP3EG) were studied in pH 7.4 PBS buffer solution, containing α-chymotrypsin at 37° C. The data from this biodegradation study showed that both polymers exhibited much faster and more pronounced weight loss in α-chymotrypsin solution than in a pure PBS buffer, and the degree of biodegradability of these PEEA polymers in an enzyme solution depended on both the chemical structures and the enzyme concentration. The biodegradation data showed that AP3EG was very sensitive towards enzymatic biodegradation (FIG. 1).

Even in a 0.05 mg/mL concentration of α-chymotrypsin solution, AP3EG easily biodegraded losing 13% of molecule weight in the first 8 hours and 31% of molecular weight after one day. The biodegradation profiles of AP3EG showed that the rate of enzymatic hydrolysis increased with an increase in the concentration of α-chymotrypsin. Moreover, irrespective of different α-chymotrypsin concentrations, the weight loss kinetics were very close to zero-order. It was also observed that the AP3EG films can maintain their constant surface area very well up to 80% of weight loss while the polymer sample becomes thinner and thinner. This result indicates that the invention PEEA polymer films underwent an even surface erosion during enzymatic hydrolysis, a result that is contrary to the type of bulk biodegradation common to aliphatic polyesters.

The effect of on biodegradability of different length of diacid monomer 1a-b in the invention OEG-based PEEAs is shown in FIG. 2. The data summarized therein indicates that the tendency of AP3EG (derived from monomer 1a: di-p-nitrophenyl adipate with 4 methylene groups) to undergo α-chymotrypsin catalyzed hydrolysis is lower than that of SP3EG (derived from monomer 1b: di-p-nitrophenyl sebacate with 8 methylene groups). In a 0.10 mg/mL α-chymotrypsin solution, SP3EG also showed biodegradation kinetics close to zero-order, and an even faster rate of biodegradation than that of AP3EG, 81% vs 56% weight loss within 24 hrs. This result may be attributed to the more hydrophobic PEEA, such as SP3EG, having a greater affinity for α-chymotrypsin and hence a higher rate of enzymatic hydrolysis than AP3EG.

These OEG-based PEEAs also showed much higher rates of α-chymotrypsin catalyzed biodegradation than similarly structured PEAs derived from linear aliphatic saturated and unsaturated diols. For example, within 24 hrs, weight loss for SP3EG was 81.3%, while weight loss for SPBU (from 2-butene-1,4-diol) was 13.8% and for SPB (derived from 1,4-butanediol) was 6.4% only. Therefore, incorporation of ether linkages into the backbones of OEG-based PEEA enhances enzymatic biodegradation as compared with that of similarly structured PEAs having aliphatic backbone segments (i.e. derived from aliphatic diols).

TABLE 2
Weigh loss, molecular weight and contact
angle of PEEA polymers before and
after incubation in different media for 18 hrs

| | Conc. of α- | | | | | |
|---|---|---|---|---|---|---|
| | chymotrypsin | Incubation | Weight | Mn | Mw | |
| Polymer | (mg/mL) | time (hr) | Loss (%) | (kg/mol) | (kg/mol) | Mw/Mn |
| AP3EG | N/A^{a} | 0 | 0 | 17.4 | 26.0 | 1.49 |
| | 0 (pure PBS) | 18 | −0.6 | 15.3 | 24.7 | 1.61 |
| | 0.05 | 18 | 25.1 | 17.4 | 25.5 | 1.46 |
| | 0.10 | 18 | 34.9 | 18.2 | 26.2 | 1.44 |
| | 0.20 | 18 | 62.3 | 17.6 | 25.7 | 1.46 |
| SP3EG | N/A^{a} | 0 | 0 | 27.3 | 1.1 | 1.51 |
| | 0 (pure PBS) | 18 | 3.4 | 28.3 | 43.5 | 1.54 |
| | 0.10 | 18 | 64.8 | 29.0 | 44.2 | 1.53 |

^{a}Original Sample as the control.

The PEEA polymer films in the incubation media were also measured by GPC to study their biodegradation (Table 2). Although the weight loss showed significant biodegradation of AP3EG (34.9%) and SP3EG (64.8%) samples in 0.1 mg/mL α-chymotrypsin solution for 18 hrs, the GPC data showed very little change in molecular weight or molecular weight distribution, and proportional results occurred in lower (0.05 mg/mL, less weight loss), higher (0.20 mg/mL, more weight loss) α-chymotrypsin concentration and in PBS buffer (almost no weight loss).

### Example 2

In this example, preparation and testing of a series of saturated OEG-based PEURs (PEEURs) is described to illustrate the properties of the invention compositions. For synthesis of key monomers of hydrophilic PEEURs, active bis-p-nitrophenyl carbonates containing ether blocks of various lengths were obtained. Active bis-p-nitrophenyl carbonate of 2EG, 3EG, and 4EG were synthesized according to general scheme VII.

In general, to a chilled (−10° C.) solution of 0.01 mole of an ether-diol, 0.02 mole of p-nitrophenyl chloroformate was added stepwise, and then the reaction temperature was increased up to ambient temperature and stirred for 1 h. The reaction solution then was refluxed until the liberation of HCl was completed and the solution was evaporated up to dryness to obtain solid product, which was further purified as follows: 1. Diethyleneglycol-bis-p-nitrophenyl carbonate (4a in Scheme VII) was recrystallized in a mixture of toluene/n-hexane (3:1 v/v). Yield 73%, m.p. 110-112° C., (lit. 108-110° C., R. Katsarava, et al., Vysokomolek. Soed. (Russia) (1987), 29A:2069); 2. The active carbonates on the basis of 3EG and 4EG (4b and 4c in Scheme VII) were recrystallized from dichloromethane/diethyl ether (3:1 v/v) mixture. Yield: 4b 60%, 4c 54%, m.p.: 4b, 59-61° C.; 4c 48-50° C.

Hydrophilic counter-partners—di-p-toluensulfonic acid salts of diamines (compound 2d-e, Scheme V) on the basis of the shortest diol (ethylene glycol) and α-amino acids of low hydrophobicity—L-alanine and L-leucine, were prepared according to the standard scheme V, as described in herein. The products were recrystallized as follows: 2e (A1EG)—from acetone/ethanol (50/1 v/v) mixture, mp=223-225° C.; and 2d (L1EG)—from acetone/water (50/1 v/v) mixture, mp=235-237° C.

The polycondensation of monomers 2d-e and 4a-c was carried out under standard conditions in DMA, in the presence of triethylamine as an acid acceptor, concentration c=1.2 mol/L, t=80° C., duration 16 h, according to Scheme VIII.

Molecular weights of the synthesized polymers (PEEURs) were within the range from 31,400 to 51,000 g/mol, with polydispersities in the range from 1.3-1.5. Polymers had a glass transition temperature (Tg) within the range from 12 to 39° C.

All of the PEEURs were soluble in DMF, chloroform, and THF. The leucine-based polymers were additionally soluble in ethanol.

### Lipase Catalyzed In Vitro Biodegradation Study.

In-vitro biodegradation of PEEURs was carried out in 0.2 N phosphate buffer with pH 7.4, t=37° C. using lipase as an enzyme. PEEURs are highly hydrophilic and films of the polymers did not retain shape in buffer solution (strong contraction was observed). Therefore, to study biodegradation, Teflon backing discs of d=4 cm were covered with the polymer by sinking the Teflon discs into the PEEUR chloroform solution, then removing and drying the polymer. This procedure was repeated until the discs attained a weight ca. 400-500 mg. Then the PEEUR-covered Teflon backing discs were dried to constant weight and used for in vitro biodegradation study. During this procedure, only 2d (L1EG)-based PEEURs remained on the backings. The much more hydrophilic 2e Ala-2 based-PEEURs flowed from the backing in buffer solution so that biodegradation by weight loss could not be studied.

The results of the lipase-catalyzed biodegradation study of 2d (L1EG)-based PEEURs are summarized in FIGS. 3A-C, in which the data show there was no noticeable extent of biodegradation in pure phosphate buffer. However, lipase-catalyzed biodegradation rates of these PEEURs was substantial and very close to the lipase-catalyzed biodegradation rate of PEA of formula I based on L-leucine, adipic acid and 1,6-hexanediol. This study showed that the length of EG fragments (EG2, EG3, or EG4) did not influence substantially the biodegradation rates of the PEEURs tested.

All publications, patents, and patent documents are incorporated by reference herein, as though individually incorporated by reference.

The invention has been described with reference to various specific and preferred embodiments and techniques. However, it should be understood that many variations and modifications might be made while remaining within the spirit and scope of the invention. Accordingly, the invention is limited only by the following claims.

What is claimed is:

1. A composition comprising at least one bioactive agent dispersed in a biodegradable polymer comprising at least one the following a) through f):
a) a poly(ester ether amide) (PEEA) having a chemical formula described by structural formula (I), 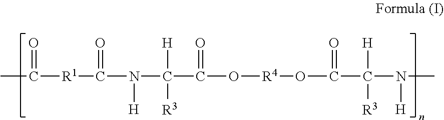
wherein n ranges from about 15 to about 150;
wherein, R¹ is independently selected from the group consisting of (C₂-C₁₂) alkylene, (C₂-C₁₂) alkenylene, and residues of α,ω-dicarboxylates of formula (II), wherein R⁵ in formula (II) is independently selected from the group consisting of (C₂-C₄) alkylene and (C₂-C₄) alkenylene and R⁷ is independently selected from the group consisting of (C₂-C₆) alkyloxy (C₂-C₁₂) alkylene, except that at least one R¹ in each polymer is the residue of a α,ω-dicarboxylate of formula (II) wherein R⁷ is (C₂-C₆) alkyloxy (C₂-C₁₂) alkylene;
R³s in individual n monomers are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂SCH₃; and
R⁴ is independently selected from (C₂-C₆) alkyloxy (C₂-C₁₂) alkylene, (C₂-C₂₀) alkylene, (C₂-C₂₀) alkenylene, CH₂CH(OH)CH₂, CH₂CH(CH₂OH), a bicyclic-fragment of a 1,4:3,6-dianhydrohexitol of structural formula (III), a fragment of 1,4-anhydroerythritol, and combinations thereof; 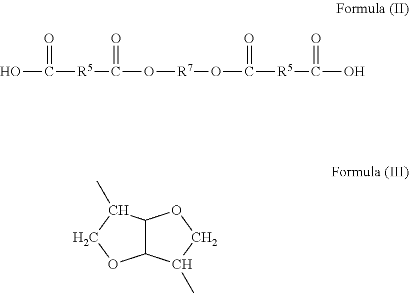
b) a PEEA polymer having a chemical formula described by structural formula (IV): 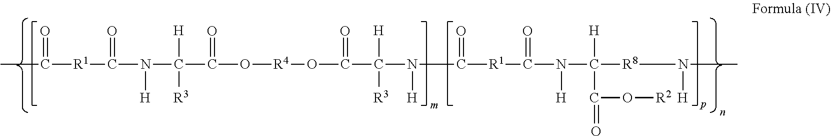
wherein n ranges from about 15 to about 150, m ranges about 0.1 to 0.9; and p ranges from about 0.9 to 0.1;
wherein R¹ is independently selected from the group consisting of (C₂-C₁₂) alkylene, (C₂-C₁₂) alkenylene, and residues of α,ω-dicarboxylates of formula (II), wherein R⁵ in formula (II) is independently selected from the group consisting of (C₂-C₄) alkylene and (C₂-C₄) alkenylene and R⁷ is independently selected from the group consisting of (C₂-C₆) alkyloxy (C₂-C₁₂) alkylene, except that at least one R¹ in each polymer is the residue of a α,ω-dicarboxylate of formula (II)) wherein R⁷ is (C₂-C₆) alkyloxy (C₂-C₁₂) alkylene;
R² is independently selected from the group consisting of hydrogen, (C₁-C₁₂) alkyl, (C₆-C₁₀) aryl (C₁-C₆) alkyl or a protecting group;
R³s in individual m monomers are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂SCH₃;
R⁴ is independently selected from (C₂-C₆) alkyloxy (C₂-C₁₂) alkylene, (C₂-C₂₀) alkylene, (C₂-C₂₀) alkenylene, CH₂CH(OH)CH₂, CH₂CH(CH₂OH), a bicyclic-fragment of a 1,4:3,6-dianhydrohexitol of structural formula (III), a fragment of 1,4-anhydroerythritol, and combinations thereof; and
R⁸ is independently (C₁-C₂₀) alkyl or (C₂-C₂₀) alkenyl;
c) a poly(ether urethane) (PEEUR) having a chemical formula described by structural formula (V), 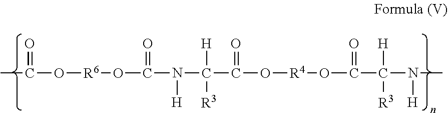
wherein, n ranges from about 15 to about 150;
R³s within an individual n monomer are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂SCH₃; and
R⁴ and R⁶ are independently selected from the group consisting of (C₂-C₆) alkyloxy (C₂-C₁₂) alkylene, CH₂CH(OH)CH₂, CH₂CH(CH₂OH), a bicyclic fragment of a 1,4:3,6-dianhydrohexitols of structural formula (III), a fragment of 1,4-anhydroerythritol and combinations thereof, except that at least one of R⁴ and R⁶ in each polymer is selected from the group consisting of (C₂-C₆) alkyloxy (C₂-C₁₂) alkylene;
d) a PEEUR having a chemical structure described by general structural formula (VI) 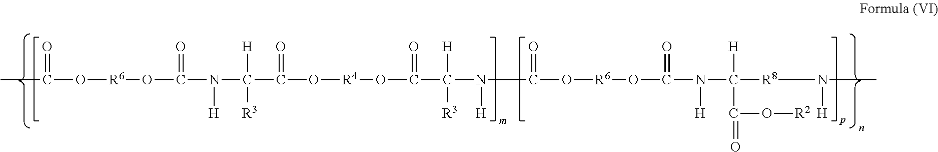
wherein n ranges from about 15 to about 150, m ranges about 0.1 to about 0.9, and p ranges from about 0.9 to about 0.1;
R² is independently selected from the group consisting of hydrogen, (C₁-C₁₂) alkyl, (C₁-C₆) alkyl (C₆-C₁₀) aryl, and a protecting group;
R³s within an individual m monomer are independently selected from the group consisting of hydrogen, (C₁-C₆) alkyl, (C₂-C₆) alkenyl, (C₂-C₆) alkynyl, (C₆-C₁₀) aryl(C₁-C₆) alkyl, and —(CH₂)₂SCH₃;
R⁴ and R⁶ are independently selected from the group consisting of (C₂-C₆) alkyloxy (C₂-C₁₂) alkylene, CH₂CH(OH)CH₂, CH₂CH(CH₂OH), a bicyclic fragment of a 1,4:3,6-dianhydrohexitol of structural formula (III), a fragment of 1,4-anhydroerythritol and combinations thereof, except that at least one of R⁴ and R⁶ in each polymer is selected from the group consisting of (C₂-C₆) alkyloxy (C₂-C₁₂) alkylene; and
R⁸ is independently (C₁-C₂₀) alkyl or (C₂-C₂₀) alkenyl;
e) a poly(ether urea) (PEEU) having a chemical formula described by general structural formula (VII): 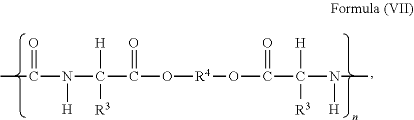
wherein n is about 15 to about 150;
R³s within an individual n monomer are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂SCH₃; and
R⁴ is independently selected from the group consisting of (C₂-C₆) alkyloxy (C₂-C₁₂) alkylene, CH₂CH(OH)CH₂, CH₂CH(CH₂OH), a bicyclic fragment of a 1,4:3,6-dianhydrohexitol of structural formula (III), a fragment of 1,4-anhydroerythritol, and combinations thereof, except that at least one R⁴ in each polymer is selected from the group consisting of (C₂-C₆) alkyloxy (C₂-C₁₂) alkylene; and
f) a PEEU having a chemical formula described by structural formula (VIII) 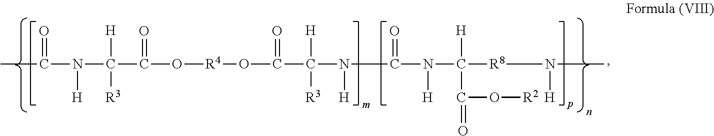
wherein m is about 0.1 to about 1.0; p is about 0.9 to about 0.1; n is about 15 to about 150;
R² is independently the group consisting of hydrogen, (C₁-C₁₂) alkyl or (C₆-C₁₀) aryl;
R³s within an individual m monomer are independently selected from the group consisting of hydrogen, (C₁-C₆) alkyl, (C₂-C₆) alkenyl, (C₂-C₆) alkynyl, (C₆-C₁₀) aryl (C₁-C₆)alkyl, and —(CH₂)₂SCH₃;
R⁴ is independently selected from the group consisting of (C₂-C₆) alkyloxy (C₂-C₁₂) alkylene, CH₂CH(OH)CH₂, CH₂CH(CH₂OH), a bicyclic fragment of a 1,4:3,6-dianhydrohexitol of structural formula (III), a fragment of 1,4-anhydroerythritol and combinations thereof, except that at least one R⁴ in each polymer is selected from the group consisting of (C₂-C₆) alkyloxy (C₂-C₁₂) alkylene; and
R⁸ is independently (C₁-C₂₀) alkyl or (C₂-C₂₀) alkenyl.

2. The composition of claim 1, wherein the polymer is described by structural formula (I) or (IV) and, in each n monomer, the R⁷ is (C₂-C₆) alkyloxy (C₂-C₁₂) alkylene.

3. The composition of claim 1, wherein the polymers are described by structural formulas (I) and (IV) and, in each n monomer, the R⁴ is independently selected from the group consisting of CH₂CH(OH)CH₂, CH₂CH(CH₂OH) and (C₂-C₆) alkyloxy (C₂-C₁₂) alkylene, and the R⁷ is (C₂-C₆) alkyloxy (C₂-C₁₂) alkylene.

4. The composition of claim 1, wherein the polymer is described by structural formula (V) or (VI), and, in each n monomer, R⁴ or R⁶ is selected from the group consisting of CH₂CH(OH)CH₂, CH₂CH(CH₂OH) and (C₂-C₆) alkyloxy (C₂-C₁₂) alkylene.

5. The composition of claim 1, wherein the polymers are described by structural formulas (V) and (VI) and, in each n monomer, R⁴ and R⁶ are selected from the group consisting of CH₂CH(OH)CH₂, CH₂CH(CH₂OH) and (C₂-C₄) alkyloxy (C₂-C₈) alkylene.

6. The composition of claim 1, wherein the polymer is described by structural formula (VII) or (VIII) and, in each n monomer, R⁴ in each n monomer is selected from the group consisting of CH₂CH(OH)CH₂, CH₂CH(CH₂OH) and (C₂-C₆) alkyloxy (C₂-C₁₂) alkylene.

7. The composition of claim 1, wherein each polymer molecule releases upon biodegradation from 15 to 300 oligo-ethylene glycol (OEG) molecules having a molecular weight of 44 up to, but not including, 400 Da.

8. The composition of claim 1, wherein the R³s are selected from the group consisting of CH₂CH(CH₃)₂, CH₂C₆H₅, and CH₃.

9. The composition of claim 1, wherein the composition is formulated for administration in the form of a liquid dispersion of particles of the polymer.

10. The composition of claim 9, wherein a particle includes from about 5 to about 150 molecules of bioactive agent per polymer molecule.

11. A polymer having a chemical formula described by structural formula (I), (IV), (V), (VI), (VII) or (VIII):
a) wherein Formula (I) is: 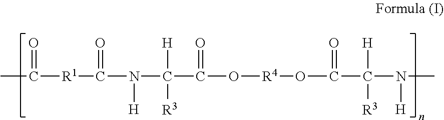
wherein n ranges from about 15 to about 150;
wherein, R¹ is independently selected from the group consisting of (C₂-C₁₂) alkylene, (C₂-C₁₂) alkenylene, and residues of α,ω-dicarboxylates of formula (II), wherein R⁵ in formula (II) is independently selected from the group consisting of (C₂-C₄) alkylene and (C₂-C₄) alkenylene and R⁷ is independently selected from the group consisting of (C₂-C₆) alkyloxy (C₂-C₁₂) alkylene, except that at least one R¹ in each polymer is the residue of a α,ω-dicarboxylate of formula (II) wherein R⁷ is independently selected from the group consisting of (C₂-C₆) alkyloxy (C₂-C₁₂) alkylene;
the R³s in individual n monomers are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂SCH₃; and
R⁴ is independently selected from (C₂-C₆) alkyloxy (C₂-C₁₂) alkylene, (C₂-C₂₀) alkylene, (C₂-C₂₀) alkenylene, CH₂CH(OH)CH₂, CH₂CH(CH₂OH), a bicyclic-fragment of a 1,4:3,6-dianhydrohexitol of structural formula (III), a fragment of 1,4-anhydroerythritol, and combinations thereof; 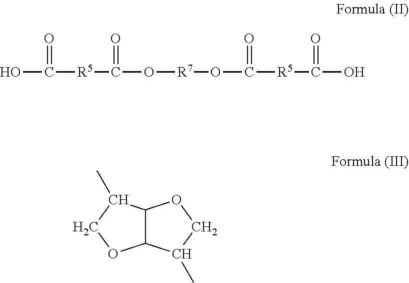
b) Formula (IV) is: 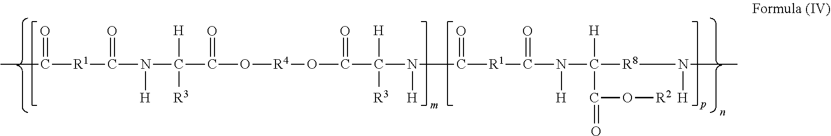
wherein n ranges from about 15 to about 150, m ranges about 0.1 to 0.9: p ranges from about 0.9 to 0.1;
wherein R¹ is independently selected from the group consisting of (C₂-C₁₂) alkylene, (C₂-C₁₂) alkenylene, and residues of α,ω-dicarboxylates of formula (II), wherein R⁵ in formula (II) is independently selected from the group consisting of (C₂-C₄) alkylene and (C₂-C₄) alkenylene and R⁷ is independently selected from the group consisting of (C₂-C₆) alkyloxy (C₂-C₁₂) alkylene, except that at least one R¹ in each polymer is the residue of a α,ω-dicarboxylate of formula (II) wherein R⁷ is independently selected from the group consisting of (C₂-C₆) alkyloxy (C₂-C₁₂) alkylene;
R² is independently selected from the group consisting of hydrogen, (C₁-C₁₂) alkyl, (C₆-C₁₀) aryl (C₁-C₆) alkyl or a protecting group;
R³s in individual m monomers are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂SCH₃;
R⁴ is independently selected from the group consisting of (C₂-C₆) alkyloxy (C₂-C₁₂) alkylene, (C₂-C₂₀) alkylene, (C₂-C₂₀) alkenylene, CH₂CH(OH)CH₂, CH₂CH(CH₂OH), a bicyclic-fragment of a 1,4:3,6-dianhydrohexitol of structural formula (III), a fragment of 1,4-anhydroerythritol, and combinations thereof; and
R⁸ is independently (C₁-C₂₀) alkyl or (C₂-C₂₀) alkenyl;
c) Formula (V) is: 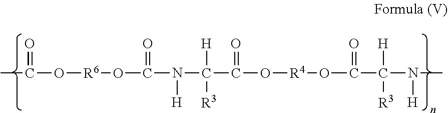
wherein n ranges from about 15 to about 150;
wherein the R³s within an individual n monomer are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂SCH₃; and
R⁴ and R⁶ are independently selected from the group consisting of (C₂-C₆) alkyloxy (C₂-C₁₂) alkylene, CH₂CH(OH)CH₂, and CH₂CH(CH₂OH), a bicyclic fragment of a 1,4:3,6-dianhydrohexitols of structural formula (III), a fragment of 1,4-anhydroerythritol and combinations thereof, except that at least one of R⁴ and R⁶ is selected from the group consisting of (C₂-C₆) alkyloxy (C₂-C₁₂) alkylene, CH₂CH(OH)CH₂, and CH₂CH(CH₂OH);
d) Formula (VI) is: 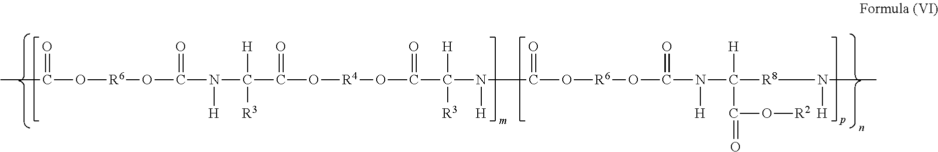
wherein n ranges from about 15 to about 150, m ranges about 0.1 to about 0.9: p ranges from about 0.9 to about 0.1;
R² is independently selected from the group consisting of hydrogen, (C₁-C₁₂) alkyl, (C₁-C₆) alkyl (C₆-C₁₀) aryl, and a protecting group;
R³s within an individual m monomer are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂SCH₃;
R⁴ and R⁶ are independently selected from the group consisting of (C₂-C₆) alkyloxy (C₂-C₁₂) alkylene, CH₂CH(OH)CH₂, CH₂CH(CH₂OH), a bicyclic fragment of a 1,4:3,6-dianhydrohexitols of structural formula (III), a fragment of 1,4-anhydroerythritol and combinations thereof, except that at least one of R⁴ and R⁶ is selected from the group consisting of (C₂-C₆) alkyloxy (C₂-C₁₂) alkylene, CH₂CH(OH)CH₂, and CH₂CH(CH₂OH); and
R⁸ is independently (C₁-C₂₀) alkyl or (C₂-C₂₀) alkenyl;
e) Formula (VII) is: 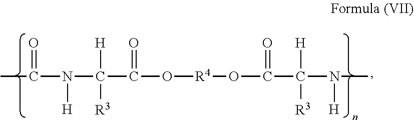
wherein n is about 15 to about 150;
the R³s within an individual n monomer are independently selected from the group consisting of hydrogen, (C₁-C₆) alkyl, (C₂-C₆) alkenyl, (C₂-C₆) alkynyl, (C₆-C₁₀) aryl (C₁-C₆) alkyl, and —(CH₂)₂SCH₃; and
R⁴ is independently selected from the group consisting of (C₂-C₆) alkyloxy (C₂-C₁₂) alkylene, CH₂CH(OH)CH₂, CH₂CH(CH₂OH; and
f) Formula (VIII) is: 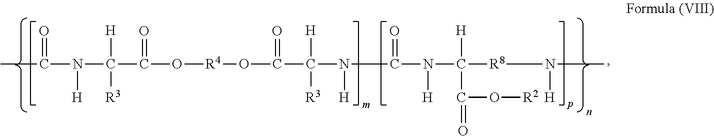
wherein m is about 0.1 to about 1.0; p is about 0.9 to about 0.1; n is about 15 to about 150;
R² is independently the group consisting of hydrogen, (C₁-C₁₂) alkyl or (C₆-C₁₀) aryl;
R³s within an individual m monomer are independently selected from the group consisting of hydrogen, (C₁-C₆) alkyl, (C₂-C₆) alkenyl, (C₂-C₆) alkynyl, (C₆-C₁₀) aryl (C₁-C₆)alkyl, and —(CH₂)₂SCH₃;
R⁴ is independently selected from the group consisting of CH₂CH(OH)CH₂, CH₂CH(CH₂OH), and (C₂-C₆) alkyloxy (C₂-C₁₂) alkylene; and
R⁸ is independently (C₁-C₂₀) alkyl or (C₂-C₂₀) alkenyl.

12. The composition of claim 11 described by structural formula (I) or (IV), wherein R⁷ is selected from the group consisting of CH₂CH(OH)CH₂, CH₂CH(CH₂OH) and (C₂-C₆) alkyloxy C₂-C₁₂) alkylene.

13. The composition of claim 11 described by structural formulas (I) and (IV), wherein R⁷s is selected from the group consisting of CH₂CH(OH)CH₂, CH₂CH(CH₂OH) and (C₂-C₄) alkyloxy (C₂-C₈) alkylene.

14. The composition of claim 11 described by structural formula (V) and (VI), wherein R⁴ and R⁶ are independently selected from the group consisting of CH₂CH(OH)CH₂, CH₂CH(CH₂OH) and (C₂-C₆) alkyloxy (C₂-C₁₂) alkylene.

15. The composition of claim 11 described by structural formula (VII) or (VIII), wherein R⁴ is selected from the group consisting of CH₂CH(OH)CH₂, CH₂CH(CH₂OH) and (C₂-C₄) alkyloxy (C₂-C₁₂)alkylene.

16. The composition of claim 11 described by structural formula (VII) and (VIII) wherein R⁴ is selected from the group consisting of CH₂CH(OH)CH₂, CH₂CH(CH₂OH) and (C₂-C₆) alkyloxy (C₂-C₁₂) alkylene.

17. The composition of claim 11, wherein the R³s are CH₂CH(CH₃)₂ or CH_{3.}

18. The composition of claim 11, wherein the R³s are selected from the group consisting of hydrogen, CH₂CH(CH₃)₂, CH₃, CH(CH₃)₂, CH(CH₃), CH₂CH₃, and CH₂C₆H₅).

19. The composition of claim 11, wherein the composition is formulated as particles with an average diameter in the range from about 10 nanometers to about 1000 microns.

20. A method for delivering a bioactive agent to a subject comprising:
administering to the subject in vivo a composition of claim 1, and
forming in vivo per polymer molecule from 15 to 300 oligo-ethylene glycol (OEG) molecules with a weight average molecular weight (Mw) of at least 44 Da but less than 400 Da, while delivering the bioactive agent to the subject at a controlled delivery rate.

21. The method of claim 20, wherein the composition biodegrades by surface enzymatic action.

22. The method of claim 20, further comprising formulating the composition as a polymer film or liquid dispersion of polymer particles prior to the administering.

23. The method of claim 22, wherein the administration involves injecting particles of the composition into a local site in the body of the subject.

24. The method of claim 20, further comprising formulating the composition into polymer particles having an average size in the range from about 1 μm to about 500 nm prior to the administration.

25. A composition comprising at least one bioactive agent dispersed in a micelle-forming polymer comprising repeating alternating units of:
a) a hydrophobic section comprising at least one polymer of claim 11, which section is joined to a water soluble section, and
b) a water soluble section of repeating alternating units of:
i) polyethylene glycol having a Mw of at least 200 Da and less than 400 Da, and
ii) at least one ionizable or polar amino acid,
wherein the repeating alternating units have substantially similar molecular weights and the Mw of the polymer is in the range from about 15 kDa to about 300 kDa.
